SEQUENCE LISTING (23 sequences)

SEQ ID NO: 1 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 2 - protein, 69 aa, Homo sapiens
NAPKRPPSAFFLFCSEYRPKIKGEHPGLSIGDVAKKLGEMWNNTAADDKQPYEKKAAKLKEKYEKDIAA

SEQ ID NO: 3 - protein, 74 aa, Homo sapiens
FKDPNAPKRPPSAFFLFCSEYRPKIKGEHPGLSIGDVAKKLGEMWNNTAADDKQPYEKKAAKLKEKYEKDIAAY

SEQ ID NO: 4 - protein, 54 aa, Homo sapiens
PDASVNFSEFSKKCSERWKTMSAKEKGKFEDMAKADKARYEREMKTYIPPKGET

SEQ ID NO: 5 - protein, 77 aa, Homo sapiens
PRGKMSSYAFFVQTCREEHKKKHPDASVNFSEFSKKCSERWKTMSAKEKGKFEDMAKADKARYEREMKTYIPPKGET

SEQ ID NO: 6 - protein, 18 aa, Homo sapiens
MGKGDPKKPRGKMSSYAF

SEQ ID NO: 7 - protein, 18 aa, Homo sapiens
YAFFVQTCREEHKKKHPD

SEQ ID NO: 8 - protein, 18 aa, Homo sapiens
HPDASVNFSEFSKKCSER

SEQ ID NO: 9 - protein, 18 aa, Homo sapiens
SERWKTMSAKEKGKFEDM

SEQ ID NO: 10 - protein, 18 aa, Homo sapiens
EDMAKADKARYEREMKTY

SEQ ID NO: 11 - protein, 18 aa, Homo sapiens
KTYIPPKGETKKKFKDPN

SEQ ID NO: 12 - protein, 18 aa, Homo sapiens
DPNAPKRPPSAFFLFCSE

SEQ ID NO: 13 - protein, 18 aa, Homo sapiens
CSEYRPKIKGEHPGLSIG

SEQ ID NO: 14 - protein, 18 aa, Homo sapiens
IKGEHPGLSIGDVAKKLG

SEQ ID NO: 15 - protein, 18 aa, Homo sapiens
SIGDVAKKLGEMWNNTAA

SEQ ID NO: 16 - protein, 18 aa, Homo sapiens
WNNTAADDKQPYEKKAAK

SEQ ID NO: 17 - protein, 18 aa, Homo sapiens
TAADDKQPYEKKAAKLKE

SEQ ID NO: 18 - protein, 18 aa, Homo sapiens
LKEKYEKDIAAYRAKGKP

SEQ ID NO: 19 - protein, 18 aa, Homo sapiens
GKPDAAKKGVVKAEKSKK

SEQ ID NO: 20 - protein, 18 aa, Homo sapiens
SKKKKEEEEDEEDEEDEE

SEQ ID NO: 21 - protein, 20 aa, Homo sapiens
DEEEEEDEEDEDEEEDDDDE

SEQ ID NO: 22 - protein, 209 aa, Homo sapiens
MGKGDPNKPRGKMSSYAFFVQTCREEHKKKHPDSSVNFAEFSKKCSERWKTMSAKEKSKFEDMAKSDKARYDREMKNYVPPKGDKKGKKKDPNAPKRPPSAFFLFCSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 23 - protein, 200 aa, Homo sapiens
MAKGDPNKPKGKTSAYAFFVQTCREEHKKKNPEVPVNFAEFSKKCSERWKTVSGKEKSKFDEMAKSDKVRYDREMKDYGPAKGGKKKKDPNAPKRPPSGFFLFCSEFRPKIKSTNPGISIGDVAKKLGEMWNNLNDSEKQPYITKAAKLKEKYEKDVADYKSKGKFDGAKGPAKVARKKVEEEDEEQEDEEEEEDEEDED

(19) United States
(12) Patent Application Publication
(10) Pub. No.: US 2008/0305120 A1
(43) Pub. Date: Dec. 11, 2008

(54) Immunogenic Compositions Comprising Hmgb 1 Polypeptides

(75) Inventors: Davorka Messmer, La Jolla, CA (US); Kevin Tracey, Old Greenwich, CT (US); Peter Kiener, Potomac, MD (US); Scott Alban, Frederick, MD (US)

Correspondence Address: MEDIMMUNE, LLC;Jonathan Klein-Evans, ONE MEDIMMUNE WAY, GAITHERSBURG, MD, 20878, US

(73) Assignees: MEDIMMUNE, INC., Gaithersburg, MD (US); THE FEINSTEIN INSTITUTE FOR MEDICAL RESEARCH, Manhasset, NY (US)

(21) Appl. No.: 11/570,695

(22) PCT Filed: Jun. 16, 2005
(86) PCT No.: PCT/US2005/021691

§ 371 Date: Aug. 5, 2008

Related U.S. Application Data
(60) Provisional application No. 60/580,549, filed on Jun. 17, 2004.

(51) Int. Cl. A61K 39/00; C07H 21/04; C12N 1/20; A61P 35/04

(52) U.S. Cl. 424/185.1; 536/23.4; 435/252.8

(57) ABSTRACT

The present invention relates to novel immunogenic compositions (e.g., vaccines), the production of such immunogenic compositions and methods of using such compositions. More specially, this invention provides unique immunogenic molecules comprising an HMGB1 polypeptide (e.g., an HMGB1 B-box polypeptide) and an antigen. Even more specifically, this invention provides novel fusion proteins comprising an isolated HMGB1 polypeptide and an antigen such that administration of these fusion proteins provides the two signals required for native T-cell activation.

### FIELD OF THE INVENTION

The present invention relates to novel immunogenic compositions (e.g., vaccines), the production of such immunogenic compositions, and methods of using such compositions. More specifically, this invention provides unique immunogenic compositions comprising an HMGB1 polypeptide (e.g., an HMGB1 B-box polypeptide) and an antigen. Even more specifically, this invention provides novel fusion proteins comprising an isolated HMGB1 polypeptide and an antigen such that administration of these fusion proteins provides the two signals required for native T-cell activation. The novel immunogenic compositions of the present invention provide an efficient way of making and using a single molecule to induce a robust T-cell immune response that activates other aspects of the adaptive immune responses. The methods and compositions of the present invention provide a powerful way of designing, producing and using immunogenic compositions (e.g., vaccines) targeted to specific antigens, including antigens associated with selected pathogens, tumors, allergens and other disease-related molecules.

### BACKGROUND OF THE INVENTION

### Innate Immunity

Multicellular organisms have developed two general systems of immunity to infectious agents. The two systems are innate or natural immunity (also known as “innate immunity”) and adaptive (acquired) or specific immunity. The major difference between the two systems is the mechanism by which they recognize infectious agents.

The innate immune system uses a set of germline-encoded receptors for the recognition of conserved molecular patterns present in microorganisms. These molecular patterns occur in certain constituents of microorganisms including: lipopolysaccharides, peptidoglycans, lipoteichoic acids, phosphatidyl cholines, bacteria-specific proteins, including lipoproteins, bacterial DNAs, viral single and double-stranded RNAs, unmethylated CpG-DNAs, mannans and a variety of other bacterial and fungal cell wall components. Such molecular patterns can also occur in other molecules such as plant alkaloids. These targets of innate immune recognition are called Pathogen Associated Molecular Patterns (PAMPs) since they are produced by microorganisms and not by the infected host organism. (Janeway et al. (1989) Cold Spring Harb. Symp. Quant. Biol. 54: 1-13; Medzhitov et al. (1997) 25 Curr. Opin. Immunol. 94: 4-9).

The receptors of the innate immune system that recognize PAMPs are called Pattern Recognition Receptors (PRRs). (Janeway et al. (1989) Cold Spring Harb. Symp. Quant. Biol. 54: 1-13; Medhitov et al. (1997) Curr. Opin. Immunol. 94: 4-9). These receptors vary in structure and belong to several different protein families. Some of these receptors recognize PAMPs directly (e.g., CD14, DEC205), while others (e.g., complement receptors) recognize the products generated by PAMP recognition. Members of these receptor families can, generally, be divided into three types: 1) humoral receptors circulating in the plasma; 2) endocytic receptors expressed on immune-cell surfaces, and 3) signaling receptors that can be expressed either on the cell surface or intracellularly. (Medzhitov et al. (1997) Curr. Opin. Immunol. 94: 4-9; Fearon et al. (1996) Science 272: 50-3). Cellular PRRs are expressed on effector cells of the innate immune system, including cells that function as professional antigen-presenting cells (APC) in adaptive immunity. Such effector cells include, but are not limited to, macrophages, dendritic cells, B-lymphocytes and surface epithelia. This expression profile allows PRRs to directly induce innate effector mechanisms, and also to alert the host organism to the presence of infectious agents by inducing the expression of a set of endogenous signals, such as inflammatory cytokines and chemokines, as discussed below. This latter function allows efficient mobilization of effector forces to combat the invaders.

In contrast, the adaptive immune system, which is found only in vertebrates, uses two types of antigen receptors that are generated by somatic mechanisms during the development of each individual organism. The two types of antigen receptors are the T-cell receptor (TCR) and the immunoglobulin receptor (IgR), which are expressed on two specialized cell types, T-lymphocytes and B lymphocytes, respectively. The specificities of these antigen receptors are generated at random during the maturation of lymphocytes by the processes of somatic gene rearrangement, random pairing of receptor subunits, and by a template-independent addition of nucleotides to the coding regions during the rearrangement.

Recent studies have demonstrated that the innate immune system plays a crucial role in the control of initiation of the adaptive immune response and in the induction of appropriate cell effector responses. (Fearon et al. (1996) Science 272: 50-3; Medzhitov et al. (1997) Cell 91: 295-8). Indeed, it is now well established that the activation of naive T-lymphocytes requires two distinct signals: one is a specific antigenic peptide recognized by the TCR, and the other is the so called co-stimulatory signal, B7, which is expressed on APCs and recognized by the CD28 molecule expressed on T-cells. (Lenschow et al. (1996) Annul Rev. Immunol. I a,: 233-58). Activation of naive CD4+ T-lymphocytes requires that both signals, the specific antigen and the B7 molecule, are expressed on the same APC. If a naive CD4 T-cell recognizes the antigen in the absence of the B7 signal, the T-cell will die by apoptosis. Expression of B7 molecules on APCs, therefore, controls whether or not the naive CD4 T-lymphocytes will be activated. Since CD4 T-cells control the activation of CD8 T-cells for cytotoxic functions, and the activation of B-cells for antibody production, the expression of B7 molecules determines whether or not all adaptive immune response will be activated.

Recent studies have also demonstrated that the innate immune system plays a crucial role in the control of B7 expression. (Fearon et al. (1996) Science 272: 50-3; Medzlitov et al. (1997) Cell 91: 295-8). As mentioned earlier, innate immune recognition is mediated by PRRs that recognize PAMPs. Recognition of PAMPs by PRRs results in the activation of signaling pathways that control the expression of a variety of inducible immune response genes, including the genes that encode signals necessary for the activation of lymphocytes, such as B7, cytokines and chemokines. (Medzhitov et al. (1997) Cell 91: 295-8; Medzhitov et al. (1997) Nature 388: 394 15 397). Induction of B7 expression by PRR upon recognition of PAMPs thus accounts for self/nonself discrimination and ensures that only T-cells specific for microorganism-derived antigens are normally activated. This mechanism normally prevents activation of autoreactive lymphocytes specific for self-antigens.

Receptors of the innate immune system that control the expression of B7 molecules and cytokines have recently been identified. (Medzhitov et al. (1997) Nature 388: 394-397; Rock et al. (1998) Proc. Natl. Acad. Sci. USA, 95: 588-93). These receptors belong to the family of Toll-like receptors (TLRs), so called because they are homologous to the Drosophila Toll protein which is involved both in dorsalventral patterning in Drosophila embryos and in the immune response in adult flies. (Lemaitre et al. (1996) Cell 86: 973-83). In mammalian organisms, such TLRs have been shown to recognize PAMPs such as the bacterial products LPS, peptidoglycan, and lipoprotein. (Schwandner et al. (1999) J; Biol. Chem. 274: 17406-9; Yoshimura et al. (1999) J. Immunol. 163: 1-5; Aliprantis et al. (1999) Science 285: 736-9).

### Vaccine Development

Vaccines have traditionally been used as a means to protect against disease caused by infectious agents. However, with the advancement of vaccine technology, vaccines have been used in additional applications that include, but are not limited to, control of mammalian fertility, modulation of hormone action, and prevention or treatment of tumors.

The primary purpose of vaccines used to protect against a disease is to induce immunological memory to a particular pathogen. More generally, vaccines are needed to induce an immune response to specific antigens, whether they arise from a particular pathogen or expressed by tumor cells or other diseased or abnormal cells. Division and differentiation of B- and T-lymphocytes that have surface receptors specific for the antigen generates both specificity and memory.

In order for a vaccine to induce a protective immune response, it must fulfill the following requirements: 1) it must include the specific antigen(s) or fragment(s) thereof that will be the target of protective immunity following vaccination; 2) it must present such antigens in a form that can be recognized by the immune system, e.g. a form resistant to degradation prior to immune recognition; and 3) it must activate APCs to present the antigen to CD4+ T-cells, which in turn induce B-cell differentiation and other immune effector functions.

Conventional vaccines contain suspensions of attenuated or killed microorganisms, such as viruses or bacteria, incapable of inducing severe infection by themselves, but capable of inducing an immune response to counteract the unmodified (or virulent) species when inoculated into a host. Usage of the term has now been extended to include essentially any preparation intended for active immunologic prophylaxis (e.g., preparations of killed microbes of virulent strains or living microbes of attenuated (variant or mutant) strains; microbial, fungal, plant, protozoan, or metazoan derivatives or products; synthetic vaccines). Examples of vaccines include, but are not limited to, cowpox virus for inoculating against smallpox, tetanus toxoid to prevent tetanus, whole-inactivated bacteria to prevent whooping cough (pertussis), polysaccharide subunits to prevent streptococcal pneumonia, and recombinant proteins to prevent hepatitis B. Although attenuated vaccines are usually immunogenic, their use has been limited because their efficacy generally requires specific, detailed knowledge of the molecular determinants of virulence. Moreover, the use of attenuated pathogens in vaccines is associated with a variety of risk factors that in most cases prevent their safe use in humans.

The problem with synthetic vaccines, on the other hand, is that they are often non-immunogenic or non-protective. The use of available adjuvants to increase the immunogenicity of synthetic vaccines is often not an option because of unacceptable side effects induced by the adjuvants themselves.

An adjuvant is defined as any substance that increases the immunogenicity of admixed antigens. Although chemicals such as alum are often considered to be adjuvants, they are in effect akin to carriers and are likely to act by stabilizing antigens and/or promoting their interaction with antigen-presenting cells. The best adjuvants are those that mimic the ability of microorganisms to activate the innate immune system. Pure antigens do not induce an immune response because they fail to induce the costimulatory signal (e.g., B7.1 or B7.2) necessary for activation of lymphocytes. Thus, a key mechanism of adjuvant activity has been attributed to the induction of costimulatory signals by microbial, or microbial-like, constituents carrying PAMPs that are routine constituents of adjuvants. (Janeway et al. (1989); Cold Spring Harb. Symp. Quant. Biol., 54: 1-13). As discussed above, the recognition of these PAMPs by PRRs induces the signals necessary for lymphocyte activation (such as B7) and differentiation (effector cytokines).

Because adjuvants are often used in molar excess of antigens and thus trigger an innate immune response in many cells that do not come in contact with the target antigen, this non-specific induction of the innate immune system to produce the signals that are required for activation of an adaptive immune response produces an excessive inflammatory response that renders many of the most potent adjuvants clinically unsuitable. Alum is currently approved for use as a clinical adjuvant, even though it has relatively limited efficacy, because it is not an innate immune stimulant and thus does not cause excessive inflammation.

### HMGB1

High mobility group box 1 (HMGB1; also known as HMG-1 and HMG1) is a protein that was first identified as the founding member of a family of DNA-binding proteins termed high mobility group box (HMGB) proteins that are critical for DNA structure and stability. It was identified nearly 40 years ago as a ubiquitously expressed nuclear protein that binds double-stranded DNA without sequence specificity. The HMGB1 protein has three domains: two DNA binding motifs termed HMGB A and HMGB B boxes, and an acidic carboxyl terminus. The two HMGB boxes are highly conserved 80 amino acid, L-shaped domains.

Recent evidence has implicated HMGB1 as a mediator of a number of inflammatory conditions (See, U.S. Pat. Nos. 6,448,223, 6,468,533, 6,303,321, which are incorporated by reference herein). HMGB1 has been demonstrated to be a long-searched-for nuclear danger signal passively released by necrotic, as opposed to apoptotic cells that will induce inflammation. Furthermore, HMGB1 can also be actively secreted by stimulated macrophages or monocytes in a process requiring acetylation of the molecule, which enables translocation from the nucleus to secretory lysosomes. HMGB1 passively released from necrotic cells and HMGB1 actively secreted by inflammatory cells are thus molecularly different. Therapeutic administration of HMGB1 antagonists rescues mice from lethal sepsis, even when initial treatment is delayed for 24 h after the onset of infection, establishing a clinically relevant therapeutic window that is significantly wider than for other known cytokines. Id.

Extracellular HMGB1 acts as a potent mediator of the inflammatory cascade by signaling via the Receptor for Advanced Glycated End-products (RAGE) and via members of the Toll-like receptor family. See, e.g., U.S. patent application no. US20040053841, which is incorporated by reference herein.

Since the initial discovery of HMGB1 as a mediator of the inflammatory cascade, it has been determined that the HMGB1 subdomains (i.e., the A-box and B-box) have distinct functional attributes. In particular, the HMG A box serves as a competitive inhibitor of HMG proinflammatory action, and the HMG B box has the predominant proinflammatory activity of HMG (see, e.g., International publication WO02092004, which is incorporated by reference herein).

### SUMMARY OF THE INVENTION

The instant invention is based, in part, on the fact that the inventors have discovered that 1) HMGB1 polypeptides (e.g., B box polypeptides) induce phenotypic maturation of dendritic cells (DCs); and 2) HMGB1 polypeptides (e.g., B box polypeptides) also induced secretion of IL-12 from DCs as well as IL-2 and IFN-γ secretion from allogeneic T cells, thus HMBG1 polypeptides function as a Th1 polarizing stimulus. The present inventors also determined that the magnitude of the induction of DC maturation by the HMGB1 polypeptide is equivalent to DCs activated by exposure to PAMPs such as LPS, non-methylated CpG oligonucleotides, or CD40L.

In addition, the inventors have discovered novel immunogenic composition comprising HMGB1 (e.g., B box) fusion polypeptides. In particular, HMGB1 polypeptides fused to a particular antigen will induce a robust specific immune response thereby increasing the immunogenicity of antigens while minimizing unnecessary inflammation, for example, at the site of vaccine injection. The antigens that would be useful to fuse to HMGB1 polypeptides include, but are not limited to pathogen-related antigens, tumor-related antigens, allergy-related antigens, neural defect-related antigens, cardiovascular disease antigens, rheumatoid arthritis-related antigens, other disease-related antigens, hormones, pregnancy related antigens, embryonic antigens and/or fetal antigens and the like).

Upon administration of a immunogenic compositions containing a HMGB1 fusion polypeptide of the invention into human or animal subjects, the HMGB1 polypeptide portion of the fusion polypeptide will interact with APCs, such as dendritic cells and macrophages. This interaction will have two consequences: First, the HMGB1 portion of the fusion will interact with a PRR such as a TLR (e.g., TLR2) and stimulate a signaling pathway, such as the NF-KB, JNK and/or p38 pathways. Second, due to the HMGB1's temporal interaction with TLRs and/or other pattern-recognition receptors, the antigen portion of the fusion polypeptide will be readily and efficiently taken up into dendritic cells and macrophages by phagocytosis, endocytosis, or macropinocytosis, depending on the cell type, the size of the fusion, and the amino acid sequence of the HMGB1 polypeptide.

Activation of TLR-induced signaling pathways will lead to the induction of the expression of cytokines, chemokines, adhesion molecules, and co-stimulatory molecules by dendritic cells and macrophages and, in some cases, B-cells. Uptake of the HMGB1-Ag fusion will lead to the processing of the antigen(s) fused to the HMGB1 polypeptide and their presentation by the MHC class-I and MHC class-II molecules. This will generate the two signals required for the activation of naive T-cells—a specific antigen signal and the co-stimulatory signal. In addition, chemokines induced by the vaccine (due to B-box interaction with TLR) will recruit naive T-cells to the APC and cytokines, like IL-12, which will induce T-cell differentiation into Th-1 effector cells. As a result, a robust T-cell immune response will be induced, which will in turn activate other aspects of the adaptive immune responses, such as activation of antigen-specific B-cells and macrophages.

Thus, the novel immunogenic compositions of the present invention provide an efficient way of making and using a single molecule to induce a robust T-cell immune response to one or more specific antigens without the adverse side effects (e.g., excessive local inflammation) normally associated with conventional vaccines. In particular, the immunogenic compositions described herein have advantages over previously described vaccines that contain antigens and adjuvant-like molecules (e.g., PAMPs) that are not fused together.

The invention is further directed to immunogenic compositions comprising an HMGB1 polypeptide (e.g., a B box polypeptide) and an antigen that are not fused together.

### BRIEF DESCRIPTION OF FIGURES

FIG. 1. HMGB1 as well as the HMGB1 B box induce phenotypic maturation of DCs. A) FACS analysis of immature DCs cultured in the presence of medium (dotted line), or increasing amounts of rHMGB1: 0.1 (interrupted line), 1.0 (thin line), or 10.0 μg/ml (thick line). DCs were analyzed for expression of the indicated markers by surface membrane immunofluorescence techniques using PE- or FITC-conjugated mAbs. B) Immature DCs were cultured with either rB box (100 μg/ml), LPS (100 ng/ml), CyC (see Methods), non-methylated CpG oligonucleotides (CpG), CD40L, or were left untreated (medium). Phenotypic maturation of DCs was assessed as above. Results represent mean +/−SEM of three independent experiments using DCs generated from different donors.

FIG. 2. B box enhances the secretion of cytokines and chemokines in DCs. (A) Immature DCs were cultured in the presence of (i) vector at 100 μg/ml, (ii) rB box at 10 or 100 μg/ml, or (iii) LPS at 10 ng/ml. Polymyxin B (200 U/ml) was added to all cultures except those treated with LPS. One representative example of four experiments is shown. (B) Immature DCs were cultured with either rB box (100 μg/ml), LPS (100 ng/ml), CyC, non-methylated CpG oligonucleotides (CpG), CD40L or left untreated (IM). Secreted cytokine levels were measured by ELISA. Results represent mean +/−SEM of three independent experiments using DCs generated from different donors.

FIG. 3. B box-induced DC maturation is not due to LPS. (A) Immature DCs were cultured in the presence of 100 μg/ml B box+polymyxin B (B box+PM), 10 ng/ml LPS, 10 ng/ml LPS+polymyxin B (LPS+PM), or treated solely with polymyxin B (IM+PM). ELISA was used to analyze cell culture supernatants for TNFα. The results shown are mean +/−SEM of three independent experiments using DCs generated from different donors. (B) Immature DCs were cultured in the presence of 10 ng/ml LPS, 10 ng/ml LPS+polymyxin B (LPS+PM), or treated with polymyxin B (IM+PM) alone. Surface membrane expression of CD83 was analyzed by FACS. The results shown are mean +/−SEM of two independent experiments using DCs generated from different donors. (C) Immature DCs were cultured in the presence of 100 μg/ml of rB box (BB) or 100 μg/ml of trypsin-digested rB box (B box-trypsin) or were left untreated (IM+PM). Polymyxin B (100 U/ml) was added to all cultures. IL-8 levels were analyzed by ELISA. The results shown are mean +/−SEM of three independent experiments using DCs generated from different donors. (D) Immature DCs were cultured in the presence of (i) medium, (ii) rB box (100 μg/ml), (iii) peptide aa 106-123 (Hp106, SEQ ID NO:13), (iv) peptide aa 121-138 (Hp121, SEQ ID NO:15), (v) peptide aa 136-153 (Hp136, SEQ ID NO:17), and (vi) peptide aa 151-168 (Hp151, SEQ ID NO:18). The amino acid sequences are numbered based on the primary HMGB1 sequence. The peptides were added at 200 μg/ml. One of three representative experiments is shown. ELISA was used to analyze IL-6 levels in culture medium. E) Immature DCs were stimulated with HMGB1 peptide aa 106-123 at 0.02-200 μg/ml, with 200 μg/ml peptide and 200 U/ml of polymyxin B (200+PM), or polymyxin B alone (medium+PM). IL-6 levels were measured by ELISA. The results shown are mean +/−SEM of two independent experiments using DCs generated from different donors. F) Immature DCs were cultured in the presence of B box (100 mg/ml), LPS (100 ng/ml), GST-control (vector) or left untreated (medium). 48 h after activation, surface expression of CD83 by DCs was measured by FACS. The results shown are mean +/−SEM of two independent experiments using DCs generated from different donors.

FIG. 4. rB box-stimulated DCs enhance the proliferation of allogeneic T cells and induce a Th1 profile. (A) Immature d7 DCs were incubated for 48 h with (i) rB box (100 μg/ml), (ii) LPS (100 ng/ml), (iii) CyC, (iv) non-methylated CpG oligonucleotides or (v) trimeric CD40L. DCs were then co-cultured with 10⁵ allogeneic T cells at a DC:T cell ratio of 1:120. T cell proliferation was assessed by measuring the amount of (³H) thymidine incorporated during the last 8 h of a 5-day culture period. A representative example of 5 independent experiments is shown as mean counts per minute (cpm), +/−SEM, from triplicate cultures. (B) After a 5-day co-culture of allogeneic T cells and DCs (DC:T ratio=1:120) that had previously been activated with the same stimuli as above, cell culture supernatants were analyzed for the presence of IFN-γ by ELISA. The results shown are mean +/−SEM of three independent experiments using DCs generated from different donors.

FIG. 5. (A) Immature DCs express RAGE on the cell surface. Immature DCs were stained with either isotype control (Ig) or with unlabeled anti-RAGE antibodies and subsequently with FITC-conjugated goat anti-rabbit to detect the primary antibody. Data are shown from one representative experiment of three similar experiments using DCs from different donors. (B) rB box induces NF-κB activation. Immature DCs were cultured in the presence of CyC, LPS (100 ng/ml), non-methylated CpG oligonucleotides (CpG), rB box (100 μg/ml), or left untreated (IM) for 48 h. Nuclear extracts were analyzed for active NF-κB. Shown is a representative of two experiments. (C) Supershift analysis of NF-κB activation. Immature DCs were cultured in the presence of rB box (100 μg/ml), or left untreated (IM) for 2 h. The supershift was carried out with the indicated antibodies using nuclear extracts from B box-stimulated DCs. Specific DNA-protein complexes were verified by competing for the DNA-binding site with unlabeled “cold” probe (co). Data represent similar observations made in two independent experiments.

FIG. 6. B box causes CD38 upregulation and IL-6 secretion via a p38 dependent pathway. Immature d7 DCs were preincubated for 30 min with (i) DMSO, (ii) PD98059 at 20 (P20) or 80 (P80) μM, (iii) SB203580 at 5 (S5) or 20 (S20) μM, (iv) TPCK at 20 (T20) or 80 (T80) μM and then treated with rB box (100 μg/ml), or cultured with DMSO in the absence of B box (medium). 48 h after activation the DCs were assayed for the surface expression of CD83 by FACS and IL-6 secretion was measured by ELISA. The results shown are mean +/−SEM of three independent experiments using DCs generated from different donors.

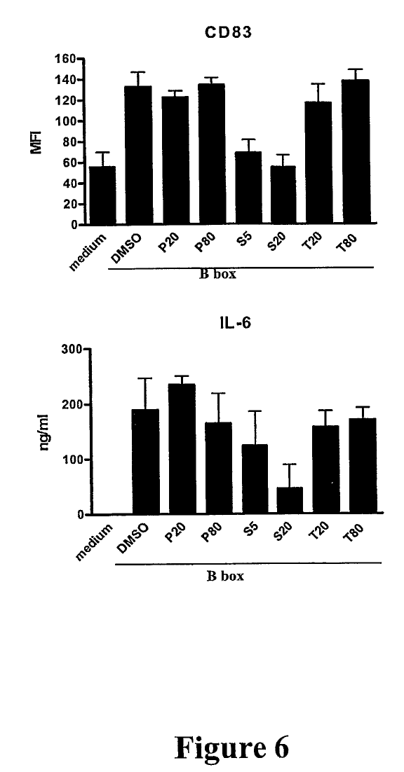

FIG. 7. Similarity comparison between the amino acid sequences of human HMGB1 (SEQ ID NO:1), HMGB2 (SEQ ID NO:22), and HMGB3 (SEQ ID NO:23).

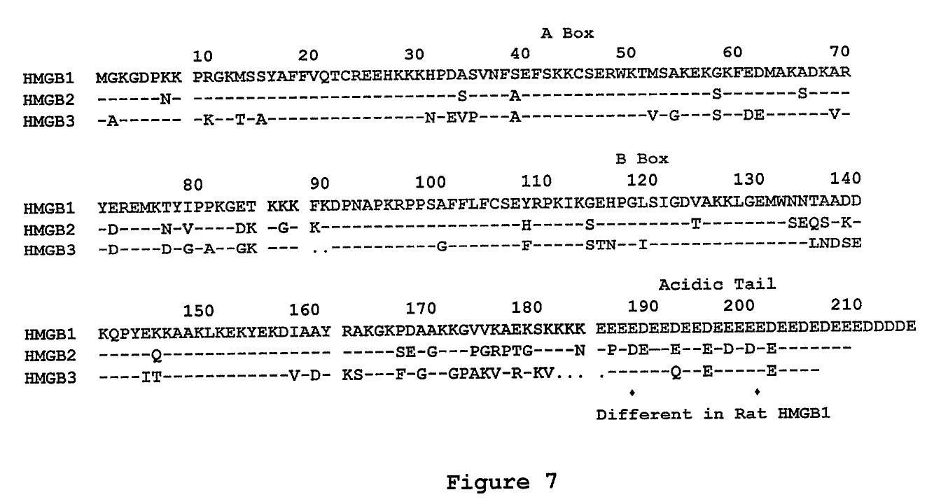

FIG. 8. HMGB1-derived peptides enhance cytokine secretion in human DCs. Secreted cytokine levels were measured by ELISA 48 h after addition of the various stimuli. Polymyxin B (200 U/ml) was added to all cultures except to the ones with LPS before addition of the stimuli. A) Immature DCs were cultured in the presence of peptides (200 μg/ml), whose sequence maps different regions of the HMGB1 molecule (For sequences see Table 1.). Results represent mean +/−SEM of two independent experiments using DCs generated from different donors. B) Immature DCs were cultured in the presence of selected peptides at (200 μg/ml), HMGB1-Bx (50 μg/ml), or left untreated (medium). All peptides are N-terminally biotinylated except 106-123 (non-bio). Results represent mean +/−SEM of three independent experiments using DCs generated from different donors. C) Immature DCs were cultured in the presence of selected peptides at (200 μg/ml), HMGB1-Bx (50 μg/ml), LPS (100 ng/ml), or left untreated (medium). Results represent mean +/−SEM of two independent experiments using DCs generated from different donors.

FIG. 9. HMGB1-Bx and HMGB1 peptides enhance the secretion of cytokines and chemokines in murine BM-DCs. Secreted cytokine levels were measured by ELISA 48 h after addition of the various stimuli. Polymyxin B (200 U/ml) was added to all cultures except to the ones with LPS before addition of the peptides. A) Immature BM-DCs were cultured in the presence of HMGB1 peptides at (200 μg/ml), HMGB1-Bx (50 μg/ml), LPS (100 ng/ml), or left untreated (medium). Results represent mean +/−SEM of three independent experiments. B) Immature BM-DCs were cultured either with HMGB1 peptides (200 μg/ml) or left untreated (medium). All peptides except 106-123 (non-bio) were N-terminally biotinylated. Results represent mean +/−SEM of three independent experiments.

FIG. 10. Phenotypic maturation of murine BM-DCs is induced by a HMGB1 peptide. FACS analysis of immature DCs cultured in the presence of either HMGB1-Bx (50 μg/ml), HMGB1 peptides (200 μg/ml), LPS (100 ng/ml), or left untreated (medium) for 48 h. DCs were gated on CD11c⁺ cells and analyzed for expression of the indicated markers by surface membrane immunofluorescence techniques using FITC-conjugated mAbs. One representative of three experiments is depicted.

FIG. 11. HMGB1-Bx- and HMGB1 peptide-stimulated murine BM-DCs enhance the proliferation of allogeneic T cells. A) Immature BM-DCs generated from C57/BL6 mice were incubated for 48 h with either HMGB1-Bx (50 μg/ml), HMGB1 peptides (200 μg/ml), or left untreated (medium). DCs were then co-cultured with 10⁵ allogeneic T cells at a DC:T cell ratio of 1:100. T cell proliferation was assessed by measuring the amount of (³H) thymidine incorporated during the last 8 h of a 5-day culture period. A representative example of 3 independent experiments is shown as mean counts per minute (cpm), +/−SEM, from triplicate cultures. B) Immature BM-DCs generated from Balb/c mice were incubated for 48 h with either HMGB1-Bx (50 μg/ml), LPS (100 ng/ml), or left untreated (medium). Their T cell stimulatory capacity was assessed as above. The data is shown as mean counts per minute (cpm), +/−SEM, from triplicate cultures.

### DETAILED DESCRIPTION OF THE INVENTION

In a preferred embodiment, the invention is directed to an immunogenic composition comprising an HMGB1 polypeptide (e.g., full-length protein, fragments thereof including the A and B box fragments and biologically active fragments thereof, and variants) and an antigen.

In addition, preferred embodiments of the invention include immunogenic compositions comprising an HMGB1 polypeptide (e.g., full-length protein, fragments, and variants) fused to a heterologous antigen. (hereinafter, HMGB1 fusion(s) of the invention” or simply “HMGB fusions” or “HMGB fusion polypeptides”).

In another preferred embodiment, immunogenic compositions comprise an HMGB1 B-box (also referred to herein as “HMGB1-Bx”) polypeptide and functional variants thereof fused to a heterologous antigen (hereinafter, “immunogenic compositions of the invention,” “B-box fusion polypeptide(s) of the invention” or simply “B-box fusions” or “B-box fusion polypeptides”).

Preferred embodiments of the invention additionally include polynucleotides that comprise or alternatively consist of polynucleotides that encode immunogenic compositions of the invention. Further, preferred embodiments of the invention include polypeptides that comprise or alternatively consist of HMGB/A-box/B-box fusion polypeptides.

It is specifically contemplated that immunogenic compositions of the invention may further comprise one or more of the following: an adjuvant, a pharmaceutically acceptable carrier.

Preferred embodiments of the invention include methods of vaccinating an animal (e.g., a mammal, a human) comprising administering the immunogenic compositions of the invention.

Preferred embodiments of the invention include methods of treating or preventing a disease (e.g., cancer or infection) comprising administering the immunogenic compositions of the invention to an animal.

Preferred embodiments of the invention further include methods of stimulating or increasing an immune response in an individual by administering an HMGB1 (e.g., an HMGB1 B-box polypeptide) polypeptide-antigen fusion to an individual, said methods comprising administering an immunogenic composition of the invention in an amount sufficient to stimulate or increase said immune response.

Preferred embodiments of the invention further include methods of stimulating or increasing an immune response in an individual by administering an HMGB1 (e.g., an HMGB1 B-box polypeptide) polypeptide-antigen fusion to an individual, said methods comprising administering an immunogenic composition of the invention in an amount sufficient to stimulate or increase said immune response, but causing less inflammation in said individual as compared to administering a HMGB1 polypeptide and the same antigen separately (i.e., unfused) or as compared to administering another adjuvant (or adjuvant-like molecule, e.g., a PAMP) and the same antigen separately (i.e., unfused).

Additional embodiments of the invention include methods of activating (in vivo, ex vivo or in vitro) APCs (e.g., DCs) comprising administering the immunogenic compositions of the invention.

Preferred antigens of the invention that may be fused to the HMGB1 polypeptides of the invention include, but are not limited to tumor, bacterial and viral antigens.

### HMGB1 Polypeptides of the Invention

HMGB1 polypeptides of the invention include full-length HMGB1 polypeptides (e.g., see, U.S. Pat. Nos. 6,448,223, 6,468,533, 6,303,321, WO02092004) and fragments and variants thereof. The amino acid sequence of full-length human HMGB1 is as follows:

(SEQ ID NO:1)

MGKGDPKKPRGKMSSYAFFVQTCREEHKKKHPDASVNFSEFSKKCSERWK
TMSAKEKGKFEDMAKADKARYEREMKTYIPPKGETKKKFKDPNAPKRPPS
AFFLFCSEYRPKIKGEHPGLSIGDVAKKLGEMWNNTAADDKQPYEKKAAK
LKEKYEKDIAAYRAKGKPDAAKKGVVKAEKSKKKKEEEEDEEDEEDEEEE
EDEEDEDEEEDDDDE.

HMGB1 polypeptides of the invention are preferably identical to, or at least 99% identical, or at least 95% identical, or at least 90% identical, or at least 85% identical, or at least 80% identical, or at least 70% identical to the human HMGB1 polypeptide shown as SEQ ID NO:1.

In a preferred embodiment, the amino acid sequences of human, rat, and mouse HMGB1 B box polypeptide are defined by either of the following sequences:

(SEQ ID NO:2)

NAPKRPPSAFFLFCSEYRPKIKGEHPGLSIGDVAKKLGEMWNNTAADDKQ
PYEKKAAKLKEKYEKDIAA
and

(SEQ ID NO:3)

FKDPNAPKRPPSAFFLFCSEYRPKIKGEHPGLSIGDVAKKLGEMWNNTAA
DDKQPYEKKAAKLKEKYEKDIAAY.

In particular, HMGB1 polypeptides of the invention include HMGB1 B box polypeptides identical to, or at least 99% identical, or at least 95% identical, or at least 90% identical, or at least 85% identical, or at least 80% identical, or at least 70% identical to the human HMGB1 B box polypeptide shown as SEQ ID NO:2 (or fragments thereof) or SEQ ID NO:3 (or fragments thereof).

In a preferred embodiment, the amino acid sequences of human, rat, and mouse HMGB1 A box polypeptide are defined by either of the following sequences:

(SEQ ID NO:4)

PDASVNFSEFSKKCSERWKTMSAKEKGKFEDMAKADKARYEREMKTYIPP
KGET
and

(SEQ ID NO:5)

PRGKMSSYAFFVQTCREEHKKKHPDASVNFSEFSKKCSERWKTMSAKEKG
KFEDMAKADKARYEREMKTYIPPKGET.

In particular, HMGB1 polypeptides of the invention include HMGB1 A box polypeptides identical to, or at least 99% identical, or at least 95% identical, or at least 90% identical, or at least 85% identical, or at least 80% identical, or at least 70% identical to the human HMGB1 A box polypeptide shown as SEQ ID NO:4 (or fragments thereof) or SEQ ID NO:5 (or fragments thereof).

In another embodiment, HMGB1 polypeptides of the invention include the sequences listing in table 1.

TABLE 1
HMGB1 peptides

| | | | |
|---|---|---|---|
| Peptide | Sequence | SEQ ID NO. | HMGB1 aa# |

| | | | |
|---|---|---|---|
| Hp-1 | MGKGDPKKPRGKMSSYAF | 6 | 1-18 |
| Hp-16 | YAFFVQTCREEHKKKHPD | 7 | 16-33 |
| Hp-31 | HPDASVNFSEFSKKCSER | 8 | 31-48 |
| Hp-46 | SERWKTMSAKEKGKFEDM | 9 | 46-63 |
| Hp-61 | EDMAKADKARYEREMKTY | 10 | 61-78 |
| Hp-76 | KTYIPPKGETKKKFKDPN | 11 | 76-93 |
| Hp-91 | DPNAPKRPPSAFFLFCSE | 12 | 91-108 |
| Hp-106 | CSEYRPKIKGEHPGLSIG | 13 | 106-123 |
| Hp-113 | IKGEHPGLSIGDVAKKLG | 14 | 113-130 |
| Hp-121 | SIGDVAKKLGEMWNNTAA | 15 | 121-138 |
| Hp-133 | WNNTAADDKQPYEKKAAK | 16 | 133-150 |
| Hp-136 | TAADDKQPYEKIKAAKLKE | 17 | 136-153 |
| Hp-151 | LKEKYEKDIAAYRAKGKP | 18 | 151-168 |
| Hp-166 | GKPDAAKKGVVKAEKSKK | 19 | 166-183 |
| Hp-181 | SKKKKEEEEDEEDEEDEE | 20 | 181-198 |
| Hp-196 | DEEEEEDEEDEDEEEDDDDE | 21 | 196-215 |

In particular, HMGB1 polypeptides of the invention include HMGB1 polypeptides identical to, or at least 99% identical, or at least 95% identical, or at least 90% identical, or at least 85% identical, or at least 80% identical, or at least 70% identical to the HMGB1 polypeptide shown in Table 1 as SEQ ID NOS:6 to 21 (or fragments thereof).

HMGB1 polypeptides of the invention may or may not be acetylated.

Other examples of the HMG A and B box polypeptides of the invention are shown in International Patent Publications WO2002092004 and WO2004046338, incorporated herein by reference. Other examples of HMGB polypeptides that contain HMGB B-box polypeptides of the invention are described in GenBank Accession Numbers CAG33144, AAH67732, AAH66889, AAH30981, AAH03378, AAA64970, AAB08987, P07155, AAA20508, S29857, P09429, NP_{—}002119, CAA31110, S02826, U00431, X67668, NP_{—}005333, NM_{—}016957, and J04179, the entire teachings of which are incorporated herein by reference. Additional examples of HMGB polypeptides that contain HMGB B-box polypeptides of the invention, include, but are not limited to mammalian HMG1 ((HMGB1) as described, for example, in GenBank Accession Number U51677), HMG2 ((HMGB2) as described, for example, in GenBank Accession Number M83665), HMG-2A ((HMGB3, HMG-4) as described, for example, in GenBank Accession Numbers NM_{—}005342 and NP_{—}005333), HMG14 (as described, for example, in GenBank Accession Number P05114), HMG17 (as described, for example, in Genbank Accession Number X13546), HMGI (as described, for example, in GenBank Accession Number L17131), and HMGY (as described, for example, in GenBank Accession Number M23618); nomnammalian HMG T1 (as described, for example, in GenBank Accession Number X02666) and HMG T2 (as described, for example, in GenBank Accession Number L32859) (rainbow trout); HMG-X (as described, for example, in GenBank Accession Number D30765) (Xenopus), HMG D (as described, for example, in GenBank Accession Number X71138) and HMG Z (as described, for example, in GenBank Accession Number X71139) (Drosophila); NHP10 protein (HMG protein homolog NHP 1) (as described, for example, in GenBank Accession Number Z48008) (yeast); non-histone chromosomal protein (as described, for example, in GenBank Accession Number 000479) (yeast); HMG ½ like protein (as described, for example, in GenBank Accession Number Z11540) (wheat, maize, soybean); upstream binding factor (UBF-1) (as described, for example, in GenBank Accession Number X53390); PMS1 protein homolog 1 (as described, for example, in GenBank Accession Number U13695); single-strand recognition protein (SSRP, structure-specific recognition protein) (as described, for example, in GenBank Accession Number M86737); the HMG homolog TDP-1 (as described, for example, in GenBank Accession Number M74017); mammalian sex-determining region Y protein (SRY, testis-determining factor) (as described, for example, in GenBank Accession Number X53772); fungal proteins: mat-1 (as described, for example, in GenBank Accession Number AB009451), ste 11 (as described, for example, in GenBank Accession Number x53431) and Mc 1; SOX 14 (as described, for example, in GenBank Accession Number AF107043) (as well as SOX 1 (as described, for example, in GenBank Accession Number Y13436), SOX 2 (as described, for example, in GenBank Accession Number Z31560), SOX 3 (as described, for example, in GenBank Accession Number X71135), SOX 6 (as described, for example, in GenBank Accession Number AF309034), SOX 8 (as described, for example, in GenBank Accession Number AF226675), SOX 10 (as described, for example, in GenBank Accession Number AJ001183), SOX 12 (as described, for example, in GenBank Accession Number X73039) and SOX 21 (as described, for example, in GenBank Accession Number AF107044)); lymphoid specific factor (LEF-1) (as described, for example, in GenBank Accession Number X58636); T-cell specific transcription factor (TCF-1) (as described, for example, in GenBank Accession Number X59869); MTT1 (as described, for example, in GenBank Accession Number M62810); and SP100-HMG nuclear autoantigen (as described, for example, in GenBank Accession Number U36501).

HMGB1 polypeptides of the invention include HMGB1 fragments preferably comprising or alternatively consisting of at least 8, or at least 15, or at least 20, or at least 25, or at least 30, or at least 50, or at least 75, or at least 100 amino acids of human HMGB1 polypeptide.

Preferred polypeptide fragments of the invention comprise or alternatively consist of an amino acid sequence selected from the following: CSEYRPKIKGEHPGLSIG (SEQ ID NO:13), DPNAPKRPPSAFFLFCSE (SEQ ID NO: 12) and IPDASVNFSEFSKKCSER (SEQ ID NO:8). In addition, biologically active fragments of the HMGB1 B box comprise or alternatively consist of an amino acid sequence selected from the following: amino acids 31-48 (SEQ ID NO: 8), 91-108 (SEQ ID NO. 12), 106-123 (SEQ ID NO:13), 121-138 (SEQ ID NO:15), and 136-153 (SEQ ID NO:17) of SEQ ID NO:1.

HMGB1 polypeptides of the invention further include functional equivalents and variants of HMGB1 polypeptides, HMGB1 A box and HMGB1 B box polypeptides. Functional equivalents of HMGB1 polypeptides, HMGB1 A box and HMGB1 B box polypeptides (proteins or polypeptides that have one or more of the biological activities (e.g., induce maturation of DCs) of an HMGB1 polypeptide, HMGB1 A box or HMGB1 B box polypeptide) can also be used in the methods of the present invention. Biologically active fragments, sequence variants, and post-translational modifications are examples of functional equivalents of a protein. Variants include a substantially homologous polypeptide encoded by the same genetic locus in an organism, i.e., an allelic variant, as well as other splicing variants.

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al., Science, 244:1081-1085 (1989)). The latter procedure introduces single alanine mutations at every residue in the molecule. The resulting mutant molecules are then tested for biological activity in vitro. Sites that are critical for polypeptide activity can also be determined by structural analysis such as crystallization, nuclear magnetic resonance or photoaffinity labeling (Smith et al., J. Mol. Biol., 224:899-904 (1992); de Vos et al., Science, 255:306-312 (1992)).

HMGB1 polypeptide, HMGB1 A box and HMGB1 B box functional equivalents also encompass polypeptides having a lower degree of identity but having sufficient similarity so as to perform one or more of the same functions performed by an HMGB1 polypeptide, HMGB1 A box or HMGB1 B box polypeptide. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al., Science, 247:1306-1310 (1990).

HMGB1 polypeptide, HMGB1 A box and HMGB B box functional equivalents also include polypeptide fragments of HMGB1, HMGB1 A box and HMGB1 B box. Fragments can be derived from an HMGB1, HMGB1 A box and HMGB1 B-box polypeptide or variants thereof. As used herein, a fragment comprises at least 6 contiguous amino acids. Useful fragments include those that retain one or more of the biological activities of the polypeptide. Examples of HMGB biologically active fragments include, for example, the first 20 amino acids of the B box (e.g., the first 20 amino acids of SEQ ID NO:1; SEQ ID NO:8; SEQ ID NO: 12 and SEQ ID NO:13). Biologically active fragments can be peptides which are, for example, at least 6, 9, 12, 15, 16, 20, 30, 35, 36, 37, 38, 39, 40, 50, 100 or more amino acids in length.

Polynucleotide sequences of the invention comprise or alternatively consist of polynucleotides that encode HMGB polypeptides of the invention. In addition, polynucleotide sequences of the invention comprise or alternatively consist of polynucleotides that encode HMGB1 fusion polypeptides, HMGB1 A box fusion polypeptides and B box fusion polypeptides of the invention.

### Antigens of the Invention

“Antigen” refers to a substance that is specifically recognized by the antigen receptors of the adaptive immune system. Thus, as used herein, the term “antigen” includes antigens, derivatives or portions of antigens that are immunogenic and immunogenic molecules derived from antigens. Preferably, the antigens used in the present invention are isolated antigens. Antigens that are particularly useful in the present invention include, but are not limited to, those that are pathogen-related, allergen-related, or disease-related.

The antigens used in the immunogenic compositions of the present invention can be any type of antigen (e.g., including but not limited to pathogen-related antigens, tumor-related antigens, allergy-related antigens, neural defect-related antigens, cardiovascular disease antigens, rheumatoid arthritis-related antigens, other disease-related antigens, hormones, pregnancy-related antigens, embryonic antigens and/or fetal antigens and the like).

In addition, the immunogenic compositions of the present invention can further comprise any PAMP peptide or protein, including, but not limited to, the following PAMPs: peptidoglycans, lipoproteins and lipopeptides, Flagellins, outer membrane proteins (OMPs), outer surface proteins (OSPs), other protein components of the bacterial cell walls, and other PRR ligands.

Examples of antigens include, but are not limited to, (1) microbial-related antigens, especially antigens of pathogens such as viruses, fungi or bacteria, or immunogenic molecules derived from them; (2) “self’ antigens, collectively comprising cellular antigens including cells containing normal transplantation antigens and/or tumor-related antigens, RR-Rh antigens and antigens characteristic of, or specific to particular cells or tissues or body fluids; (3) allergen-related antigens such as those associated with environmental allergens (e.g., grasses, pollens, molds, dust, insects and dander), occupational allergens (e.g., latex, dander, urethanes, epoxy resins), food (e.g., shellfish, peanuts, eggs, milk products), drugs (e.g., antibiotics, anesthetics) and (4) vaccines (e.g., flu vaccine).

Antigen processing and recognition of displayed peptides by T-lymphocytes depends in large part on the amino acid sequence of the antigen rather than the three-dimensional structure of the antigen. Thus, the antigen portion used in the vaccines of the present invention can contain epitopes or specific domains of interest rather than the entire sequence. In fact, the antigenic portions of the vaccines of the present invention can comprise one or more immunogenic portions or derivatives of the antigen rather than the entire antigen. Additionally, the immunogenic compositions of the present invention can contain an entire antigen with intact three-dimensional structure or a portion of the antigen that maintains a three-dimensional structure of an antigenic determinant, in order to produce an antibody response by B-lymphocytes against a spatial epitope of the antigen.

### Pathogen-Related Antigens.

Specific examples of pathogen-related antigens include, but are not limited to, antigens selected from the group consisting of vaccinia, avipox virus, turkey influenza virus, bovine leukemia virus, feline leukemia virus, avian influenza, human influenza (including but not limited to pandemic strains), chicken pneumovirosis virus, canine parvovirus, equine 41 influenza, FHV, Newcastle Disease Virus (NDV), Chicken/Pennsylvania/l/83 influenza virus, parainfluenza influenza virus (PIV), human metapneumovirus (hMPV), infectious bronchitis virus; Dengue virus, measles virus, Rubella virus, pseudorabies, Epstein-Barr Virus, HIV, SIV, EHV, BHV, HCMV, Hantaan, C. tetani, mumps, Morbillivirus, Herpes Simplex Virus type 1, Herpes Simplex Virus 5 type 2, Human cytomegalovirus, Hepatitis A Virus, Hepatitis B Virus, Hepatitis C Virus, Hepatitis E Virus, Coronavirus, Respiratory Syncytial Virus (e.g., F protein), Human Papilloma Virus, Influenza Virus (e.g., HA and NA proteins), Salmonella, Neisseria, Borrelia, Chlamydia, Bordetella, and Plasmodium and Toxoplasma, Cryptococcus, Streptococcus, Staphylococcus, Haentophilus, Diptheria, Tetanus, Pertussis, Escherichia, Candida, Aspergillus, Entamoeba, Giardia, and Trypanasonia. Numerous pathogen-related antigens are well known in the art and include for example, but not to by way of limitation, those listed below.

Some representative examples of polypeptide antigens of HIV include, but are not limited to, Gag, Pol, Vif and Nef (Vogt et al., 1995, Vaccine 13: 202-208); HIV antigens gp120 and gp160 (Achour et al., 1995, Cell. Mol. Biol. 41: 395-400; Hone et al., 1994, Dev. Biol. Stand. 82: 159-162); gp41 epitope of human immunodeficiency virus (Eckhart et al., 1996, J. Gen. Virol. 77: 2001-2008) derived from an HIV isolate selected from the group including but not limited to: HXB2, LAV-1, NY5, BRU, SF2. These references list preferred polypeptide antigens of the invention and are incorporated by reference herein.

Some representative examples of polypeptide antigens of HCV include, but are not limited to, nucleocapsid protein in a secreted or a nonsecreted form, core protein (pC); E1 (pE1), E2 (pE2) (Saito et al., 1997, Gastroenterology 112: 1321-1330), NS3, NS4a, NS4b and NS5 (Chen et al., 1992, Virology 188:102-113), derived from an HCV isolate selected from the group including but not limited to: genotypes 1a, 1b, 2a, 2b and 3a-11a.

Antigenic peptides of RSV, HMPV and PUV detailed in: Young et al., in Patent publication WO04010935A2 the teachings of which is incorporated herein by reference in its entirety. Antigenic peptides of SARS corona virus include but are not limited to, the S (spike) glycoprotein, small envelope protein E (the E protein), the membrane glycoprotein M (the M protein), the hemagglutinin esterase protein (the HE protein), and the nucleocapsid protein (the N-protein) See, e.g., Marra et al., “The Genome Sequence of the SARS-Associated Coronavirus,” Science Express, May 2003; BCCA Genome Sciences Centre, GenBank Accession no. NC_{—}004718 (May 2003); GenBank Accession Nos. AY278554, AY278491, and AY278488. These references list preferred polypeptide antigens of the invention and are incorporated by reference herein.

### Cancer-Related Antigens

The methods and compositions of the present invention can also be used to produce immunogenic compositions directed against tumor-associated protein antigens such as melanoma-associated antigens, mammary cancer-associated antigens, colorectal cancer-associated antigens, prostate cancer-associated antigens and the like.

Specific examples of tumor-related or tissue-specific protein antigens useful in such immunogenic compositions include, but are not limited to, the following antigens:

Prostate: prostate-specific antigen (PSA), prostate-specific membrane antigen (PSMA), PAP, PSCA (PNAS 95(4) 1735-1740 1998), prostate mucin antigen (PMA) (Beckett and Wright, 1995, Int. J. Cancer 62: 703-710), Prostase, Her-2neu, SPAS-1; Melanoma: TRP-2, tyrosinase, Melan A/Mart-1, gplOO, BAGE, GAGE, GM2 ganglioside; Breast: Her2-neu, kinesin 2, TATA element modulatory factor 1, tumor protein D52, MAGE D, ING2, HIP-55, TGF-1 anti-apoptotic factor, HOM-MeI-40/SSX2, epithelial antigen (LEA 135), DF31MUC1 antigen (Apostolopoulos et al., 1996 Immunol. Cell. Biol. 74: 457-464; Pandey et al., 1995, Cancer Res. 55: 4000-4003); Testis: MAGE-1, HOM-Mel-40/SSX2, NY-ESO-1; Colorectal: EGFR, CEA; Lung: MAGE D, EGFR Ovarian Her-2neu; Baldder: transitional cell carcinoma (TCC) (Jones et al., 1997, Anticancer Res. 17: 685-687), Several cancers: Epha2, Epha4, PCDGF, HAAH, Mesothelin; EPCAM; NY-ESO-1, glycoprotein MUC1 and NIUC10 mucins p5 (especially mutated versions), EGFR; Miscellaneous tumor: cancer-associated serum antigen (CASA) and cancer antigen 125 (CA 125) (Kierkegaard et al., 1995, Gynecol. Oncol. 59: 251-254), the epithelial glycoprotein 40 (EGP40) (Kievit et al., 1997, Int. J. Cancer 71: 237-245), squamous cell carcinoma antigen (SCC) (Lozza et al., 1997 Anticancer Res. 17: 525-529), cathepsin E (Mota et al., 1997, Am. J Pathol. 150: 1223-1229), tyrosinase in melanoma (Fishman et al., 1997 Cancer 79: 1461-1464), cell nuclear antigen (PCNA) of cerebral cavemomas (Notelet et al., 1997 Surg. Neurol. 47: 364-370), a 35 kD tumor-associated autoantigen in papillary thyroid carcinoma (Lucas et al., 1996 Anticancer Res. 16: 2493-2496), CDC27 (including the mutated form of the protein), antigens triosephosphate isomerase, 707-AP, A60 mycobacterial antigen (Maes et al., 1996, J. Cancer Res. Clin. Oncol. 122: 296-300), Annexin II, AFP, ART-4, BAGE, β-catenin/m, BCL-2, bcr-abl, bcr-abl p190, bcr-abl p210, BRCA-1, BRCA-2, CA 19-9 (Tolliver and O'Brien, 1997, South Med. J. 90: 89-90; Tsuruta et al., 1997 Urol. Int. 58: 20-24), CAMEL, CAP-1, CASP-8, CDC27/m, CDK-4/m, CEA (Huang et al., Exper Rev. Vaccines (2002) 1:49-63), CT9, CT10, Cyp-B, Dek-cain, DAM-6 (MAGE-B2), DAM-10 (MAGE-B1), EphA2 (Zantek et al., Cell Growth Differ. (1999) 10:629-38; Carles-Kinch et al., Cancer Res. (2002) 62:2840-7), EphA4 (Cheng et al., 2002, Cytokine Growth Factor Rev. 13:75-85), tumor associated Thomsen-Friedenreich antigen (Dahlenborg et al., 1997, Int. J Cancer 70: 63-71), ELF2M, ETV6-AML1, G250, GAGE-1, GAGE-2, GAGE-3, GAGE-4, GAGE-5, GAGE-6, GAGE-7B, GAGE-8, GnT-V, gp100 (Zajac et al., 1997, Int. J. Cancer 71: 491-496), HAGE, HER2/neu, HLA-A*0201-R170I, HPV-E7, HSP70-2M, HST-2, hTERT, hTRT, iCE, inhibitors of apoptosis (e.g., survivin), KH-1 adenocarcinoma antigen (Deshpande and Danishefsky, 1997, Nature 387: 164-166), KIAA0205, K-ras, LAGE, LAGE-1, LDLR/FUT, MAGE-1, MAGE-2, MAGE-3, MAGE-6, MAGE-A1, MAGE-A2, MAGE-A3, MAGE-A4, MAGE-A6, MAGE-A10, MAGE-A12, MAGE-B5, MAGE-B6, MAGE-C2, MAGE-C3, MAGE-D, MART-1, MART-1/Melan-A (Kawakami and Rosenberg, 1997, Int. Rev. Immunol. 14: 173-192), MC1R, MDM-2, Myosin/m, MUC1, MUC2, MUM-1, MUM-2, MUM-3, neo-polyA polymerase, NA88-A, NY-ESO-1, NY-ESO-1a (CAG-3), PAGE-4, PAP, Proteinase 3 (Molldrem et al., Blood (1996) 88:2450-7; Molldrem et al., Blood (1997) 90:2529-34), P15, p190, Pm1/RARα, PRAME, PSA, PSM, PSMA, RAGE, RAS, RCAS1, RU1, RU2, SAGE, SART-1, SART-2, SART-3, SP17, SPAS-1, TEL/AML1, TPI/m, Tyrosinase, TARP, TRP-1 (gp75), TRP-2, TRP-2/INT2, WT-1, and alternatively translated NY-ESO-ORF2 and CAMEL proteins, derived from the NY-ESO-1 and LAGE-1 genes. Numerous other cancer antigens are well known in the art.

In order for tumors to give rise to proliferating and malignant cells, they must become vascularized. Strategies that prevent tumor vascularization have the potential for being therapeutic. The methods and compositions of the present invention can also be used to produce vaccines directed against tumor vascularization. Examples of target antigens for such vaccines are vascular endothelial growth factors, integrins (e.g., alphaV beta3) vascular endothelial growth factor receptors, fibroblast growth factors and fibroblast growth factor receptors and the like.

### Allergen-Related Antigens

The methods and compositions of the present invention can be used to prevent or treat allergies and asthma. Thus, the methods and compositions of the present invention can also be used to construct immunogenic compositions that may suppress allergic reactions.

Specific examples of allergen-related protein antigens useful in the methods and compositions of the present invention include, but are not limited to: allergens derived from pollen, such as those derived from trees such as Japanese cedar (Cryptomeria, Cryptomeria japonica), grasses (Gramineae), such as orchard-grass (Dactylis, Daetylis glomerata), weeds such as ragweed (Ambrosia, Ambrosia artemisiffiblia); specific examples of pollen allergens including the Japanese cedar pollen allergens (J Allergy Clim Immunol. (1983) 71: 77-86) and (FEBS Letters (1988) 239: 329-332), and the ragweed allergens Amb a 1.1, Amba 1.2, Amb a 1.3, Amb a 1.4, Amb a I1 etc.; allergens derived from fungi (Aspergillus, Candida, 41ternaria, etc.); allergens derived from mites (allergens from Dermatophagoidespteronyssinus, Derinatophagoidesfarinae etc.; specific examples of mite allergens including Der p I, Der p II, Der p III, Der p VII, Der f I, Der f II, Der f III, Der f VII etc.); house dust; allergens derived from animal skin debris, feces and hair (for example, the feline allergen Fel d 1); allergens derived from insects (such as scaly hair or scale of moths, butterflies, Chironomidae etc., poisons of the Vespidae, such as Vespa maizdarinia); food allergens (eggs, milk, meat, seafood, beans, cereals, fruits, nuts and vegetables etc.); allergens derived from parasites (such as roundworm and nematodes, for example, Anisakis); and protein or peptide based drugs (such as insulin). Many of these allergens are commercially available.

### Other Disease Antigens.

Also contemplated in this invention are vaccines directed against antigens that are associated with diseases other than cancer, allergy and asthma. As one example of many, and not by way of limitation, an extracellular accumulation of a protein cleavage product of P-amyloid precursor protein, called “amyloid-peptide”, is associated with the pathogenesis of Alzheimer's disease. (Janus et al., Nature (2000) 408: 979-982; Morgan et al., Nature (2000) 408: 982985). Thus, the fusions used in the immunogenic compositions of the present invention can include amyloid-O peptide, or antigenic domains of amyloid-P peptide, as the antigenic portion of the construct, and a HMGB1 polypeptide.

Examples of other diseases in which vaccines might be generated against self-proteins or self peptides are the following:

Autoimmune diseases: disease-linked HLA-alleles (e.g., HLA DRB 1, HLA-DRI, HLA-DR6B I proteins or fragments thereof, chain genes); TCR chain sub-groups; CD11a (leukocyte function-associated antigen 1; LFA-1); IFNy; IL-10; TCR analogs; IgR analogs; 21-hydroxylase (for Addison's disease); calcium sensing receptor (for acquired hypoparathyroidism); tyrosinase (for vitiligo); Cardiovascular disease: LDL receptor; Diabetes: glutamic acid decarboxylase (GAD), insulin B chain; PC-1; IA-2, IA 2b; GLIMA-38; Epilepsy: NMDA C.

### Identification of Antigens.

New antigens and novel epitopes also can be identified using methods well known in the art. Any conventional method, e.g., subtractive library, comparative Northern and/or Western blot analysis of normal and tumor cells, Serial Analysis of Gene Expression (U.S. Pat. No. 5,695,937) and SPHERE (described in PCT WO 97/35035), can be used to identify putative antigens for use.

For example, expression cloning as described in Kawakami et al., 1994, Proc. Natl. Acad. Sci. 91: 3515-19, also can be used to identify a novel tumor-associated antigen. Briefly, in this method, a library of cDNAs corresponding to mRNAs derived from tumor cells is cloned into an expression vector and introduced into target cells which are subsequently incubated with cytotoxic T cells. Pools of cDNAs that are able to stimulate T Cell responses are identified and through a process of sequential dilution and re-testing of less complex pools of cDNAs, unique cDNA sequences that are able to stimulate the T cells and thus encode a tumor antigen are identified. The tumor-specificity of the corresponding mRNAs can be confirmed by comparative Northern and/or Western blot analysis of normal and tumor cells.

SAGE analysis can be employed to identify the antigens recognized by expanded immune effector cells such as CTLs, by identifying nucleotide sequences expressed in the antigen-expressing cells. SAGE analysis begins with providing complementary deoxyribonucleic acid (cDNA) from an antigen-expressing population and cells not expressing the antigen. Both cDNAs can be linked to primer sites. Sequence tags are then created, for example, using appropriate primers to amplify the DNA. By measuring the differences in these tag sets between the two cell types, sequences which are aberrantly expressed in the antigen-expressing cell population can be identified.

Another method to identify optimal epitopes and new antigenic peptides is a technique known as Solid PHase Epitope REcovery (“SPHERE”). This method is described in detail in PCT WO 97/35035. Although used to screen for MHC class I-restricted CTL epitopes, the method can be modified to screen for class II epitopes by screening for the stimulation of antigen-specific MHC class II specific T cell lines, for example, rather than CTL. In SPHERE, peptide libraries are synthesized on beads where each bead contains a unique peptide that can be released in a controlled manner. Eluted peptides can be pooled to yield wells with any desired complexity. After cleaving a percentage of the peptides from the beads, these are assayed for their ability to stimulate a Class II response, as described above. Positive individual beads are then be decoded, identifying the reactive-amino acid sequence. Analysis of all positives will give a partial profile of conservatively substituted epitopes which stimulate the T cell response being tested. The peptide can be resynthesized and retested to verify the response. Also, a second library (of minimal complexity) can be synthesized with representations of all conservative substitutions in order to enumerate the complete spectrum of derivatives tolerated by a particular response. By screening multiple T cell lines simultaneously, the search for crossreacting epitopes can be facilitated.

### HMGB1—Antigen Fusions of the Invention

HMGB1 fusion polypeptides comprise an HMGB1 polypeptide of the invention and a heterologous antigen.

A preferred HMGB1 fusion polypeptide of the invention comprises an HMGB1 B box polypeptide of the invention and a heterologous antigen. Another preferred HMGB1 fusion polypeptide of the invention comprises an HMGB1 A box polypeptide of the invention and a heterologous antigen.

HMGB1 fusion polypeptides may also comprise additional peptide sequence, e.g., linking the heterologous antigen and the HMGB1 polypeptide. A linking polypeptide can be at least 1, or at least 2, or at least 3, or at least 4, or at least 5, or at least 6, or at least 7, or at least 8, or at least 9, or at least 10, or at least 15, or at least 25, or at least 35, or at least 40, or at least 60, or at least 100 amino acids in length.

It is also specifically contemplated that additional peptide sequences can be located amino or carboxy terminal of either the HMGB1 portion or the antigen portion of the fusion.

Procedures for construction of fusion proteins are well known in the art (see e.g., Williams, et al., J. Cell Biol. 111: 955, 1990). DNA sequences encoding the desired polypeptides can be obtained from readily available recombinant DNA materials such as those available from the American Type Culture Collection, P.O. Box 1549, Manassas, Va., 20108, or from DNA libraries that contain the desired DNA.

The DNA segments corresponding to the desired polypeptide sequences (e.g., one or more HMGB1 polypeptide and an antigen) are then assembled with appropriate control and signal sequences using routine procedures of recombinant DNA methodology. See, e.g., as described in U.S. Pat. No. 4,593,002, and Langford, et al., Molec. Cell. Biol. 6: 3191, 1986 (each of which is incorporated herein).

A DNA sequence encoding a protein or polypeptide can be synthesized chemically or isolated by one of several approaches. The DNA sequence to be synthesized can be designed with the appropriate codons for the desired amino acid sequence. In general, one will select preferred codons for the intended host in which the sequence will be used for expression. The complete sequence may be assembled from overlapping oligonucleotides prepared by standard methods and assembled into a complete coding sequence. See, e.g., Edge, Nature 292: 756, 1981; Nambair, et al. Science 223: 1299, 1984; Jay, et al., J. Biol. Chem. 259: 6311, 1984. In one aspect, one or more of the nucleic acids encoding the desired polypeptide sequences of the HMGB1 protein and the antigen are isolated individually using the polymerase chain reaction (M. A. Innis, et al., In PCR Protocols: A Guide To Methods and Applications, Academic Press, 1990). The domains are preferably isolated from publicly available clones known to contain them, but they may also be isolated from genomic DNA or cDNA libraries. Preferably, isolated fragments are bordered by compatible restriction endonuclease sites which allow a fusion DNA encoding both the HMGB1 polypeptide and the antigen polypeptide sequence to be constructed. This technique is well known to those of skill in the art. Alternatively, nucleotide sequences encoding the HMGB1 and antigen polypeptide sequences may be fused directly to each other (e.g., with no intervening sequences), or inserted into one another (e.g., where the sequences are discontinuous), or may separated by intervening sequences (e.g., such as linker sequences).

The basic strategies for preparing oligonucleotide primers, probes and DNA libraries, as well as their screening by nucleic acid hybridization, are well known to those of ordinary skill in the art. Such techniques are explained fully in the literature. See, e.g., Current Protocols in Molecular Biology, F. M. Ausubel et al., ed., John Wiley & Sons (Chichester, England, 1998); Molecular Cloning: A Laboratory Manual, 3nd Edition, J. Sambrook et al., ed., Cold Spring Harbor Laboratory Press (Cold Spring Harbor, N.Y., 2001); DNA Cloning: A Practical Approach, Volumes I and II (D. N. Glover, ed., 1985); Oligonucleotide Synthesis (M. J. Gait, ed., 1984); Nucleic Acid Hybridization (B. D. Hames & S. J. Higgins, eds., 1985); Transcription and Translation (B. D. Hames & S. I. Higgins, eds., 1984); Animal Cell Culture (R. I. Freshney, ed., 1986); Immobilized Cells and Enzymes (IRL Press, 1986); B. Perbal, A Practical Guide to Molecular Cloning (1984), each of which is incorporated herein in its entirety. The construction of an appropriate genomic DNA or cDNA library is within the skill of the art. See, e.g., Perbal, 1984, supra. Alternatively, suitable DNA libraries or publicly available clones are available from suppliers of biological research materials, such as Clonetech and Stratagene, as well as from public depositories such as the American Type Culture Collection.

Selection may be accomplished by expressing sequences from an expression library of DNA and detecting the expressed peptides immunologically. These selection procedures are well known to those of ordinary skill in the art (see, e.g., Sambrook, et al., 2001, supra). Once a clone containing the coding sequence for the desired polypeptide sequence has been prepared or isolated, the sequence can be cloned into any suitable vector, preferably comprising an origin of replication for maintaining the sequence in a host cell.

HMGB1 fusion polypeptides (e.g., HMGB1 B box fusion polypeptides) may also specifically comprise a peptide sequence or other modification which increases the stability of the fusion. The additional polypeptide does not necessarily need to be directly fused (i.e., produced as part of the fusion protein), but may be fused through linker sequences. Such polypeptides include, for example, human serum albumin, PEGylation and L-amino acids.

HMGB1 fusion polypeptides (e.g., HMGB1 B box fusion polypeptides) of the invention may incorporate one or more polypeptide modifications and/or conjugates selected for their ability to increase the stability, biological half-life, or other biological or manufacturing property. Such modifications and conjugates include, but are not limited to, biotinylation, acetylation, glycosylation, phosphorylation, myristylation, prenylation, ribosylation, carboxylation, pegylation radiolabels, as well as, other biochemical and chemical modifications known to one of skill in the art.

HMGB1 fusion polypeptides (e.g., HMGB1 B box fusion polypeptides) may also comprise two or more antigens.

HMGB1 fusion polypeptides (e.g., HMGB1 B box fusion polypeptides) may also comprise two or more distinct HMGB1 polypeptides (see, e.g., Table 1). For example, a single HMGB1 fusion polypeptide may comprise in addition to at least one antigen, amino acids 31-48 (SEQ ID NO: 8) and 91-108 (SEQ ID NO. 12) corresponding to HMGB1 (SEQ ID NO:1) in the absence of the intervening HMGB1 amino acids.

HMGB1 fusion polypeptides (e.g., HMGB1 B box fusion polypeptides) may also comprise a polypeptide that facilitates purification or production, e.g., GST, His-tag, such polypeptides are often referred to as “marker amino acid sequences”. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

It is also contemplated that the HMGB1 fusion polypeptides (e.g., HMGB1 B box fusion polypeptides) may be conjugated to each other to generate dimers, trimers and even higher order structures. HMGB1 fusion polypeptides may be conjugated using chemical cross-linking methods well known in the art. Alternatively, a polynucleotide encoding multimers of HMGB1 fusion polypeptides may be generated using molecular biology methods well known in the art (see, e.g. Ausebel et al., 1998; and Sambrook et al., 2001, supra).

### Vaccine Formulation Administration

Vaccine material according to this invention may contain the HMGB1 fusion polypeptides of the invention (e.g., HMGB1 B box fusion polypeptides) or may be recombinant microorganisms, or antigen presenting cells which express the HMGB1 fusion polypeptides of the invention (e.g., HMGB1 B box fusion polypeptides). Vaccines may also be prepared which contain polynucleotides encoding the HMGB1 fusion polypeptides of the invention. Preparation of compositions containing vaccine material according to this invention and administration of such compositions for immunization of individuals are accomplished according to principles of immunization that are well known to those skilled in the art.

Large quantities of these materials may be obtained by culturing recombinant or transformed cells containing replicons that express the HMGB1 fusion polypeptides described above (e.g., HMGB1 B box fusion polypeptides). Culturing methods are well-known to those skilled in the art and are taught in one or more of the documents cited above. The vaccine material is generally produced by culture of recombinant or transformed cells and formulated in a pharmacologically acceptable solution or suspension, which is usually a physiologically-compatible aqueous solution, or in coated tablets, tablets, capsules, suppositories or ampules, as described in the art, for example in U.S. Pat. No. 4,446,128, incorporated herein by reference. Administration may be any suitable route, including oral, rectal, intranasal or by injection where injection may be, for example, transdermal, subcutaneous, intramuscular or intravenous.

Compositions comprising the HMGB1 fusion polypeptides of the present invention may be formulated for any appropriate manner of administration, including for example, topical, oral, nasal, mucosal, intravenous, intracranial, intraperitoneal, subcutaneous and intramuscular administration.

While any suitable carrier known to those of ordinary skill in the art may be employed in the pharmaceutical compositions of this invention, the type of carrier will typically vary depending on the mode of administration.

The development of suitable dosing and treatment regimens for using the particular HMGB1 fusion polypeptide compositions described herein in a variety of treatment regimens, including e.g., oral, parenteral, intravenous, intranasal, and intramuscular administration and formulation, is well known in the art, (see for example, “Remington's Pharmaceutical Sciences” 15th Edition, pages 1035 1038 and 1570-1580; Mathiowitz et a/., Nature 1997 Mar. 27; 386(6623):410-4; Hwang et a/., Crit. Rev Ther Drug Carrier Syst 1998; 15(3):243-84; U.S. Pat. No. 5,641,515; U.S. Pat. No. 5,580,579 and U.S. Pat. No. 5,792,451, each of which is incorporated herein in its entirety.)

The HMGB1 fusion polypeptide composition is administered to a mammal in an amount sufficient to induce an immune response in the mammal. A minimum preferred amount for administration is the amount required to elicit antibody formation to a concentration at least 4 times that which existed prior to administration. A typical initial dose for administration would be 10-5000 micrograms when administered intravenously, intramuscularly or subcutaneously, or 10⁵ to 10¹¹ plaque forming units of a recombinant vector, although this amount may be adjusted by a clinician doing the administration as commonly occurs in the administration of vaccines and other agents which induce immune responses. A single administration may usually be sufficient to induce immunity, but multiple administrations may be carried out to assure or boost the response.

HMGB1 fusion polypeptide (e.g., HMGB1 B box fusion polypeptide) compositions may be tested initially in a non-human mammal (e.g., a mouse or primate). For example, assays of the immune responses of inoculated mice can be used to demonstrate greater antibody, T cell proliferation, and/or cytotoxic T cell responses to the HMGB1 fusion polypeptides than to unfused antigen. HMGB1 fusion polypeptides or DNA molecule encoding HMGB1 fusion polypeptides can be evaluated in Rhesus monkeys to determine whether such vaccine formulation that is highly effective in mice will also elicit an appropriate monkey immune response. In one aspect, each monkey receives a total of 5 mg DNA per immunization, delivered IM and divided between 2 sites, with immunizations at day 0 and at weeks 4, 8, and 20, with an additional doses optional. Antibody responses, including but not limited to, ADCC, CD4⁺ and CD8⁺ T-cell cytokine production, CD4⁺ and CD8⁺ T-cell antigen-specific cytokine staining can be measured to monitor immune responses to the HMGB1 and HMG B box fusion polypeptide compositions.

Without further description, it is believed that one of ordinary skill in the art can, using the preceding description and the following illustrative examples, practice the methods of the present invention. The following working examples are illustrative only, and are not to be construed as limiting in any way the remainder of the disclosure. Other generic and specific configurations will be apparent to those persons skilled in the art.

### EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### Example 1

The initation and control of an adaptive immune response is critical for health and disease. DCs are central to these processes (1, 2). DCs detect evolutionarily conserved molecular structures unique to foreign pathogens, such as LPS (3), DNA molecules containing unmethylated CpG motifs (4); they also respond to endogenous signals of cellular distress or damage (5-8). Interaction with these agents stimulates DCs to undergo the process of maturation. Endogenous factors that cause DCs to mature are an important class of stimuli that might contribute to the initiation or perpetuation of an immune response against pathogens. On the other hand, if these factors are released chronically and/or in the absence of infection, they could potentially contribute to the activation of self-reactive T cells and play a role in the development of autoimmunity (5, 6).

HMGB1, a nuclear and cytosolic protein, was originally identified as an intranuclear factor with an important structural function in chromatin organization (9). Recently, HMGB1 was identified as a proinflammatory cytokine that mediates endotoxin lethality, local inflammation and macrophage activation (10-12, 35). HMGB1 administered in vivo induces arthritis when injected into murine joints (13) and acute lung injury when administered intraarticularly (14, 38). HMGB1 is released by activated macrophages and monocytes following exposure to LPS, TNF-α or IL-1β and as a result of tissue damage (15, 16). It is a also a potent stimulating signal to monocytes and induces the delayed synthesis of pro-inflammatory cytokines (11). Furthermore, HMGB1 also enhances IFN-γ release from macrophage-stimulated NK cells (36). RAGE (37) as well as TLR2 and TLR4 (29) have been reported as HMGB1 receptors. HMGB1 contains two homologous DNA-binding motifs termed HMG A and HMG B boxes (17, 18). The pro-inflammatory domain of HMGB1 maps to the B box, which alone is sufficient to recapitulate the cytokine-stimulating effect of full length HMGB1 in vivo (19). The intracellular abundance of HMGB1, and its proinflammatory activities, suggest the possibility that its release at sites of cell injury or damage plays a role in the initiation and/or perpetuation of an immune response. Furthermore, since HMGB1 is found in the serum of patients with acute (sepsis) and chronic (rheumatoid arthritis) inflammatory conditions (10, 20), it may be involved in maladaptive or autoimmune responses.

It is shown that HMGB1, via its B box domain, induced phenotypic maturation of DCs as evidenced by increased CD83, CD54, CD80, CD40, CD58, and MHC-II expression and decreased CD206 expression. The B box caused increased secretion of the pro-inflammatory cytokines and IL-12, IL-6, IL-1α, IL-8, TNF-α and RANTES. B box upregulated CD83 expression as well as IL-6 secretion via a p38 mitogen activated protein kinase (MAPK) dependent pathway. In the mixed leukocyte reaction, B box-activated DCs acted as potent stimulators of allogeneic T cells and the magnitude of the response was equivalent to DCs activated by exposure to LPS, non-methylated CpG oligonucleotides, or CD40L. Furthermore, B box also induced secretion of IL-12 from DCs as well as IL-2 and IFN-γ secretion from allogeneic T cells suggesting a Th1 bias. HMGB1 released by necrotic cells may be a signal of tissue or cellular injury that, when sensed by DCs, induces and/or enhances an immune reaction.

### Materials and Methods

### Reagents

Human recombinant HMGB1 or recombinant B box (rB Box) was expressed in Escherichia coli and purified to homogeneity as described (21). Briefly, B box (233 bp) was cloned by PCR amplification from a human Brain Quick-Clone cDNA (Clontech, Palo Alto, Calif.). The primers were: 5′ (AAGTTCAAGGATCCCAATGCAAAG) 3′ and 5′ (GCGGCCGCTCAATAT GCAGCTATATCCTTTTC) 3′. PCR product was subcloned into pCRII-TOPO vector EcoR I sites using TA cloning method, per the manufacturer's instruction (Invitrogen, Carlsbad, Calif.). After amplification, the PCR product was digested with EcoR I and subcloned into an expression vector (pGEX) with a GST (glutathione S-transferase) tag (Pharmacia, Piscataway, N.J.). The recombinant plasmid was transformed into protease-deficient E. coli strain BL21 (Novagen, Madison, Wis.) and incubated in 2-YT medium containing ampicillin (50 μg/ml) for 5-7 hours at 30° C. with vigorous shaking until an OD at A₆₀₀ of 1-1.5 was achieved. Subsequently, fusion protein expression was induced by addition of 1 mM IPTG for 3 hours at 25° C. Bacteria were sonicated in ice-cold PBS plus 1× protease inhibitor cocktail (Sigma, St. Louis, Mo.) and 1 mM PMSF. After centrifugation (8,000×g) to remove bacterial debris, the rB box was purified to homogeneity by the Glutathione Sepharose resin column chromatography (Pharmacia). For use as a control in cell stimulation experiments, GST vector protein was expressed and purified similarly, and then used as the control for experiments using recombinant GST-B box protein. Protein elute was dialyzed extensively against PBS to remove excess amount of reduced glutathione, and passed over a column with immobilized polymyxin B (Pierce, Rockford, Ill.) to remove LPS. Recombinant B box purified to homogeneity, contained trace amounts of LPS (19 pg LPS/μg rB box) as measured by the chromogenic Limulus amebocyte lysate assay (BioWhittacker Inc, Walkersville, Md.). For all stimulation experiments using the rB box, polymyxin B was added to the cell culture medium at 200 U/ml, an amount that completely neutralizes the activity of these amounts of LPS.

### Inhibitors

The p38 MAPK-specific inhibitor, SB203580, a pyridinyl imidazole compound, the MEK inhibitor PD98059, and N-tosyl-L-phenylalanine chloromethyl ketone (TPCK) were purchased from Sigma. Because these required solubilization in DMSO, DMSO was used as a negative control.

### Generation of RAGE Antibodies

Polyclonal anti-RAGE antibody reactive with an extracellular region of human RAGE (peptide sequence SVKEQTRRHPETGLFTC) was raised in rabbits (Cocalica Biologicals, Inc., Reamstown, Pa.). IgG was purified using protein A agarose (Pierce).

### Trypsin Treatment of HMGB1 B Box

To proteolytically digest HMGB1 proteins, trypsin-EDTA was added to HMGB1 B box (0.05% final concentration) and digestion was carried out in 1×PBS (pH 7.4) at 25° C. overnight. Degradation of proteins was verified by SDS-PAGE before and after trypsin digestion by Coomassie blue staining.

### T Cell Isolation

T cells were isolated by negative selection using the RosefteSep antibody cocktail from StemCell Technologies (Vancouver, Calif.) according to the manufacturer's instructions. The cell purity of the isolated T cells was routinely ˜99% pure.

### Generation of DCs

PBMCs were isolated from the blood of normal volunteers (Long Island Blood Services, Melville, N.Y.) over a Ficoll-Hypaque (Amersham Biosciences, Uppsala, Sweden) density gradient. CD14⁺ monocytes were isolated from PBMCs by positive selection using anti-CD14 beads (Miltenyi Biotech., Auburn, Calif.) following the manufacturer's instructions. To generate DCs, CD14⁺ cells were cultured in RPMI 1640 medium supplemented with 2 mM L-glutamine (GIBCO-BRL Life Technologies; Grand Island, N.Y.), 50 μM 2-mercaptoethanol (Sigma, St. Louis, Mo.), 10 mM HEPES (GIBCO-BRL), penicillin (100 U/ml)-streptomycin (100 μg/ml) (GIBCO-BRL), and 5% human AB serum (Gemini Bio-Products, Woodland, Calif.). Cultures were maintained for 7 days in 6-well trays (3×10⁶ cells/well) supplemented with 1000 U GM-CSF per ml (Immunex, Seattle, Wash.) and 200 U IL-4 per ml (R&D Systems; Minneapolis, Minn.) at days 0, 2, 4 and 6.

### Stimulation of DCs

At day 7 of culture, immature DCs were either left untreated (“immature”, IM), or were stimulated with indicated doses of (i) LPS (E. coli serotype 026:B6, Sigma), (ii) 500 ng/ml of trimeric CD40L (Alexis Biochemicals; San Diego, Calif.), (iii) a cocktail of cytokines (CyC) consisting of IL-6 at 1000 U/ml, TNFα at 10 ng/ml, IL-1β at 10 ng/ml (all from R&D Systems) and prostaglandin E2 (PGE-2) at 1 μg/ml (Sigma), or with (v) non-methylated CpG oligonucleotides (5′ tcgtcgttttgtcgttttgtcgtt 3′) at 30 μg/ml. The sequence of this oligonucleotide is known to induce DC maturation (4), except that it contains an unmodified phosphodiester backbone. In all experiments, DCs were analyzed 48 h after stimulation. All experiments using the rB box were performed in the presence of polymyxin B sufficient to neutralize greater than 10-fold more LPS than present in rB box preparations.

### Analysis of DC Phenotype

1×10⁴ DCs were reacted for at least 20 min at 4° C. in 100 μl of PBS/5% FCS/0.1% sodium azide (staining buffer) with phycoerythrin (PE)-conjugated IgG specific for CD206, CD54, HLA-DR (all from Beckton Dickinson Immunostaining Systems; San Jose. Calif.) and CD83 (Immunotech-Beckman-Coulter; Marseille, France) or fluorescein isothiocyanate (FITC)— conjugated IgG mAb specific for CD80, CD40 and CD58 (all from Beckton Dickinson Immunostaining Systems; San Jose. Calif.). Cells were then washed 4 times with staining buffer, fixed in 10% formaldehyde in PBS (pH 7.2-7.4) and examined by flow cytometry using a FACScan (BD). In all experiments, isotype controls were included using an appropriate PE- or FITC-conjugated irrelevant mAb of the same Ig class.

### Measurement of Cytokines and Chemokines

48 h post activation, the production of cytokines and chemokines in cell culture supernatants was measured by ELISA (Pierce Boston Technology Center, SearchLight™ Proteome Arrays Multiplex Sample Testing Services, Woburn, Mass.).

### Mixed Leukocyte Reaction

To assess levels of cellular activation and proliferation, cells were plated at 10⁵ cells per well in a round-bottomed 96-well tray at DC:T cell ratios of 1:120 for 5 days in medium described above. The microcultures were pulsed with (³H)-thymidine (1 mCi/well) for the final 8 h of culture. Cell cultures were harvested onto glass fiber filters with an automated multiple sample harvester and the amount of isotope incorporation was determined by liquid scintillation β-emission. Responses are reported as mean cpm of thymidine incorporation by triplicate cultures (+/−SEM).

### Electrophoretic Mobility Shift Assay (EMSA)

DCs were collected 48 h after activation and washed 1× in PBS. Nuclear extract was isolated using the NE-PER Nuclear and Cytoplasmic Extraction Reagents from Pierce according to the manufacturer's instructions (Pierce Biotechnology, Rockford, Ill.). For detection of NF-κB binding, nuclear extract from cells (˜5 μg protein) was incubated with 0.2 ng of ³²P-labeled double-stranded oligonucleotide sequence in a 10 μl reaction volume containing 5× gel shift binding buffer (20% glycerol, 5 mM MgCl₂, 2.5 mM EDTA, 2.5 mM DTT, 250 mM NaCl, 50 mM Tris-HCl (pH 7.5), and 0.25 mg/ml poly(dI-dC)-poly(dI-dC)) for 30 min at RT. For supershift experiments 1 μl of antibody against the indicated NF-κB family members (Santa Cruz Biotechnology, Santa Cruz, Calif.) was added to the reaction mix 5 min before loading on the gel. The samples were resolved on a 4% polyacrylamide gel and visualized directly by autoradiography after drying the gel. The NF-κB consensus sequence (Promega, # E3292) was labeled with 10 U of T4 Polynucleotide kinase (Promega, #M4101) per 25 ng of oligo, 1× kinase buffer and 5 μL (32^{P})-γATP (Amersham, #PB10168, 10 mCi/ml) for 30 min at 37° C. Unbound ATP with a Sephadex G25 column (Boehringer Mannheim, #1273949).

### Results

### HMGB1 and the HMGB1 B Box Induce Phenotypic Maturation of DCs.

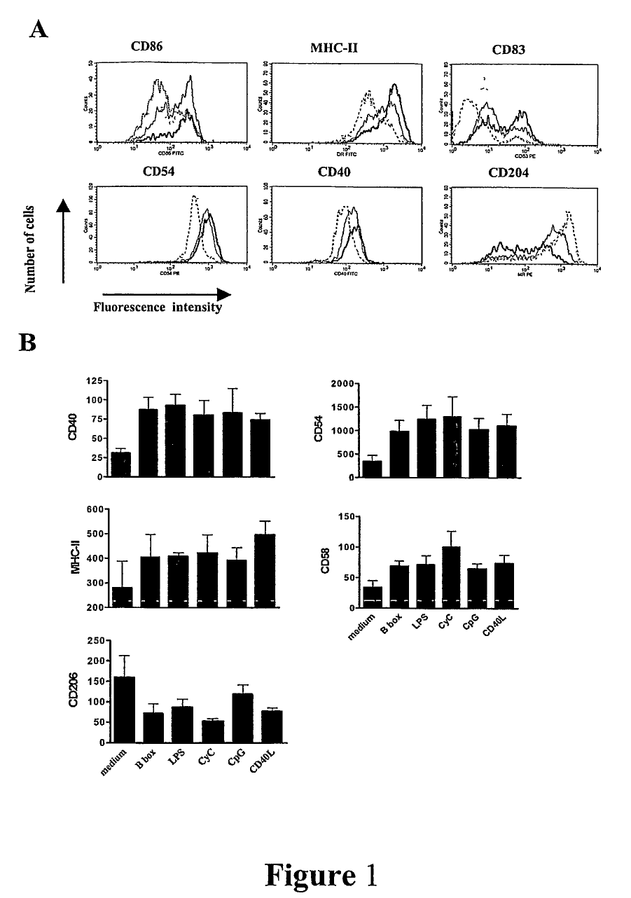

To determine whether HMGB1 can induce DC maturation, recombinant HMGB1 was added to immature DCs, and increases in CD83, MHCII, CD54, CD86, CD40 expression and decrease in CD206 expression were observed (FIG. 1.A). Previous work has linked the proinflammatory domain of HMGB1 to the B box (21). Immature DCs were exposed to rB box or other known maturation stimuli: LPS, CyC, non-methylated CpG oligonucleotides (CpG), and CD40L (see methods). rB box increased the cell surface density of CD83, CD54, CD58 CD40, and MHCII, and decreased the density of the macrophage mannose receptor, CD206. The B box-induced changes were quantitatively similar to the other stimuli (FIG. 1.B).

### The HMGB1 B Box Causes Secretion of Pro-Inflammatory Cytokines and Chemokines.

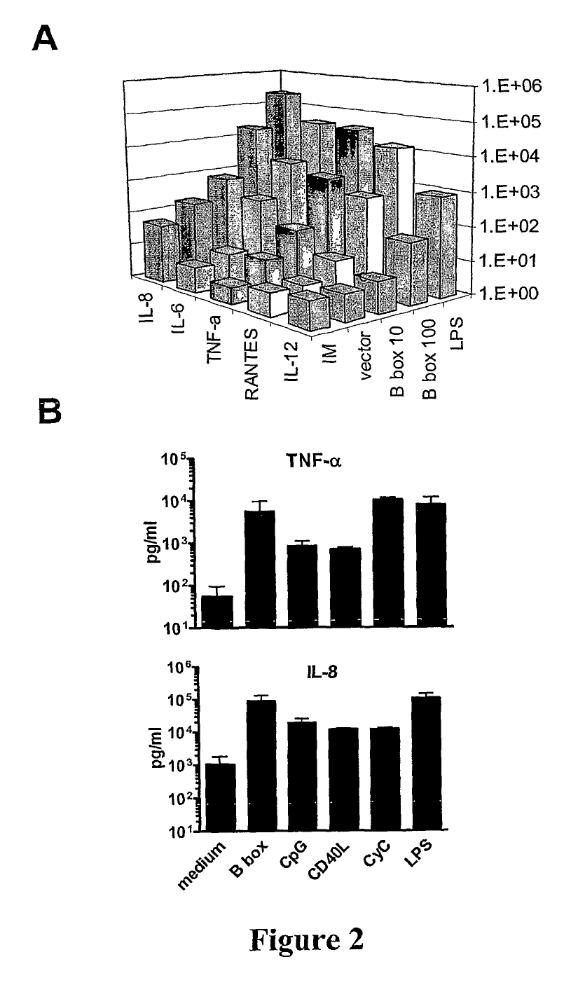

In addition to changes in surface molecule expression, secretion of inflammatory cytokines and chemokines characterizes DC maturation. rB box, when cultured with immature DCs, induced the secretion of IL-12 (p70), TNF-α, IL-6, IL-1α, RANTES, and IL-8 (FIG. 2A). IL-10 levels were either not detected or very low (<10 pg/ml). IL-2, IFN-α and TGF-β did not increase beyond detectable levels (data not shown). The GST control (see methods; vector) did not induce the secretion of these factors (FIG. 2A). Furthermore, rB box induced the secretion of IL-8, and TNF-α at levels similar to the other stimuli (FIG. 2B).

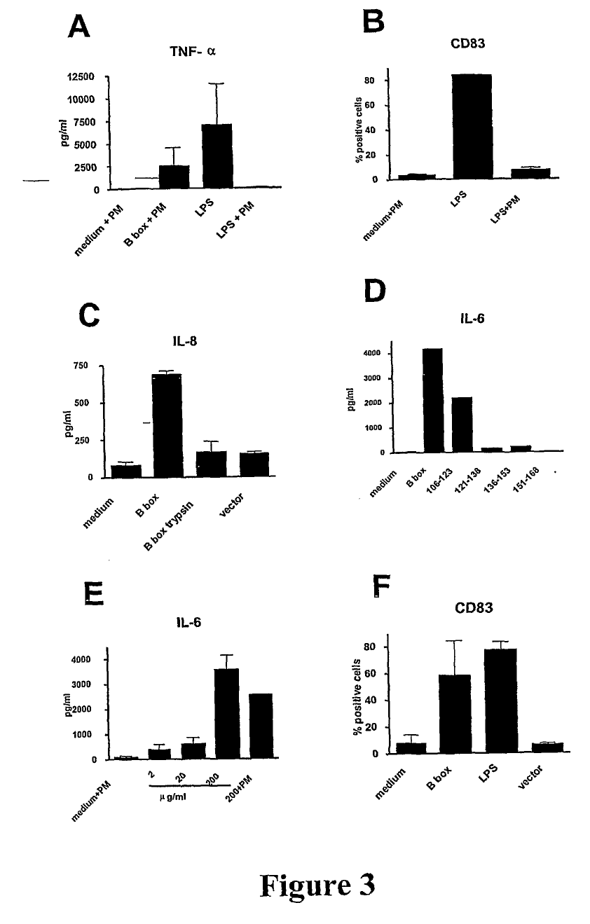

All experiments using rB box were performed in the presence of polymyxin B at concentrations sufficient to neutralize >10-fold the amount of contaminating LPS in rB box preparations (FIGS. 3A, B). Additional evidence for the specificity of these observations was obtained by digesting rB box with trypsin, which is known to abrogate the macrophage stimulatory activity of HMGB1 but has no effect on the activity of LPS (11). Neither the trypsin-treated rB box nor the recombinant protein control (GST) elicited the secretion of inflammatory cytokines (FIG. 3C). Furthermore, the GST-control did not upregulate CD83 expression (FIG. 3F).

Similar observations were made using chemically synthesized, overlapping 17aa long peptides that span the B box. The synthetic peptide CSEYRPKIKGEHPGLSIG, which corresponds to HMGB1 aa 106-123, specifically stimulated IL-6 release (FIG. 3D, E) to levels comparable to rB box. The synthetic peptides had no detectable levels of LPS; addition of polymyxin to these cultures did not alter the observed results significantly. Thus, rB box and a chemically synthesized B box peptide specifically stimulate DC maturation.

### B Box Induces Functional Maturation of DCs and Leads to a Th1 Polarized Immune Response.

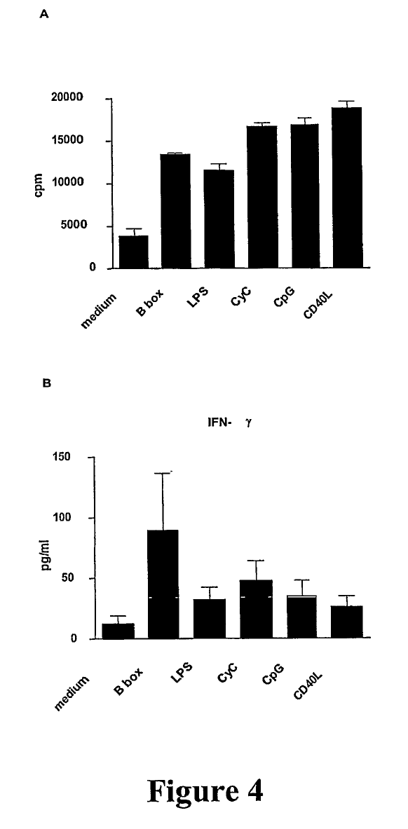

Mature, cytokine-producing DCs induce T cell activation and proliferation, leading to the development of adaptive immunity (1, 22). In co-culture experiments, DCs matured by exposure to B box, activated resting allogeneic T cells. The magnitude of this stimulation was equivalent to DCs that had been matured by exposure to LPS, CyC, non-methylated CpG oligonucleotides or with CD40L (FIG. 4A), indicating that rB box induced functional maturation of DCs. DCs matured with rB box or the other stimuli were cultured with allogeneic T cells for 5 days and subsequently the culture supernatants were analyzed for the presence of Th1 and Th2 cytokines. IFN-γ levels were the highest in the rB box-matured DC-T cell co-cultures (FIG. 4B). All stimuli upregulated IL-2 about 7-fold over immature DCs. IL-2 levels in rB box-stimulated DC cultures (51.1+/−22.4 pg/ml) were similar to the other stimuli (42.4+/−8.4 pg/ml). IL-5 was most strongly upregulated by each of the stimuli, albeit to differing levels: CyC-stimulated DCs 42-fold over immature DCs (277.4+/−242.6 pg/ml), rB box activated DCs 26-fold (143.7+/−142.4 pg/ml) and the other stimuli activated DCs 12-fold (147.2+/−30 pg/ml). IL-4 was not detected after exposure to any of the stimulants.

### rB Box Activates NF-κB.

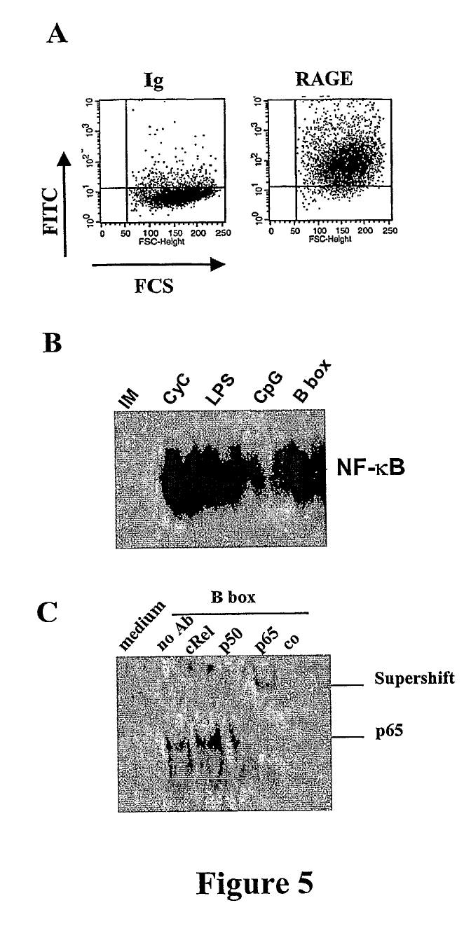

HMGB1 is a ligand for the receptor for advanced glycation endproducts (RAGE), a membrane protein that transduces intracellular signaling thereby leading to nuclear translocation of NFκB (23),(24). Using immunofluorescence techniques, RAGE was detected on the cell surface of immature DCs (FIG. 5A). Since signaling through NF-κB also plays a role in DC maturation (25-27), we tested whether B box activated NF-κB using an electrophoretic mobility shift assay (EMSA). As expected, immature DCs showed no active NF-κB (28), whereas B box-stimulated DCs expressed levels of active NF-κB comparable to CyC and LPS (FIG. 5B). To identify the NF-κB subunits involved in B box-induced intracellular signaling, nuclear extracts from B box activated DCs were analyzed by supershift assay. Anti-p65 antibody caused a significant supershift in protein/DNA complex motility, indicating that the p65 subunit is a component of the complex activated by B box (FIG. 5C). Antibodies against p52 and Rel B did not cause supershifts (data not shown). Unlabeled NF-κB-specific DNA probes displaced the NF-κB-DNA complex, indicating specificity for the NF-kB consensus sequence (FIG. 5C).

### rB Box Upregulates CD83 Expression and IL-6 Secretion in a p38 MAPK-Dependent Manner.

To obtain insight into the roles of ERK and p38 MAPK pathways in B box-induced DC maturation, we used their specific inhibitors, PD98059 and SB203580, respectively. To analyze the role of NF-κB we used TPCK, a serine protease inhibitor that blocks nuclear translocation of Rel/NF-κB by preventing IκB degradation. The p38 MAPK inhibitor SB203580, but not the other inhibitors, completely abrogated B box-induced upregulation of CD83 and downregulated B box induced secretion of IL-6 (FIG. 5).

### Example 2

As shown above (Example 1, and see, Messmer et al. (41)) HMGB1 and its B box domain are potent stimuli for maturation of human monocyte-derived DCs. Here we demonstrate that smaller peptide fragments that map to the B box domain also showed stimulatory activity on DCs. Rovere-Querini et al. (39) have recently shown that HMGB1 is a crucial component of necrotic lysates that can induce maturation of murine DCs and that it has adjuvant activity in vivo. However, it has not been investigated whether the HMGB1 alone is sufficient to induce maturation of murine DCs. While the potential for whole HMGB1 protein as adjuvant has been demonstrated there are limitations for using large recombinant proteins as adjuvants and it would be more practical and desirable to use synthetic small protein fragments or ideally peptides as adjuvants due to the uncomplicated production and purity obtained.

We show in this study that the HMGB1 B box domain, which is about a third of the size of HMGB1 is sufficient to induce phenotypic and functional maturation of murine BM-DCs. In addition several novel HMGB1-derived peptides were identified that can activate both human and murine DCs and that are attractive candidates for vaccine adjuvants. Furthermore, since these peptides induce different spectra of cytokines in DCs they could be used to generate customized DCs.

### Materials and Methods

### Reagents

Recombinant HMGB1-B box domain (HMGB1-Bx) was expressed in Escherichia coli and purified as described (21, 40). Purified HMGB1-Bx contained trace amounts of LPS (19 pg LPS/μg B box) as measured by the chromogenic Limulus amebocyte lysate assay (BioWhittacker Inc, Walkersville, Md.). Therefore, all experiments using HMGB1-Bx as well as the peptides were performed in the presence of polymyxin B (200 U/ml) sufficient to neutralize >10-fold the amount of contaminating LPS in HMGB1-Bx preparations. We have previously shown that the DC stimulatory capacity of HMGB1-Bx requires an intact tertiary structure and is not due to contaminating amounts of LPS, as trypsinization abolished HMGB1-Bx activity (41).

### Animals

Female C57/BL6 and Balb/c mice, 6-8 weeks of age, were purchased from The Jackson Laboratory (Bar Harbor, Me.) and housed at the North Shore-LIJ Research Institute's animal facility. All animals studies were approved by the Animal Subjects Conunittee and the biosafety committee at the North Shore-LIJ Research Institute and were performed in accordance with institutional guidelines.

### Peptides

All peptides were synthesized with an N-terminal biotin. The peptides are named by their first amino acid in the HMGB1 sequence. The peptide sequences are listed in Table 1.

### T Cell Isolation

T cells were isolated by negative selection using the mouse SpinSep antibody cocktail from StemCell Technologies (Vancouver, Calif.) according to the manufacturer's instructions. The cell purity of the isolated T cells was routinely ˜99% pure.

### Generation of Human DCs

PBMCs were isolated from the blood of normal volunteers (Long Island Blood Services, Melville, N.Y.) over a Ficoll-Hypaque (Amersham Biosciences, Uppsala, Sweden) density gradient. CD14⁺ monocytes were isolated from PBMCs by positive selection using anti-CD14 beads (Miltenyi Biotech., Auburn, Calif.) following the manufacturer's instructions. To generate DCs, CD14⁺ cells were cultured in RPMI 1640 medium supplemented with 2 mM L-glutamine (GIBCO-BRL Life Technologies; Grand Island, N.Y.), 50 μM 2-mercaptoethanol (Sigma, St. Louis, Mo.), 10 mM HEPES (GIBCO-BRL), penicillin (100 U/ml)-streptomycin (100 μg/ml) (GIBCO-BRL), and 5% human AB serum (Gemini Bio-Products, Woodland, Calif.). Cultures were maintained for 7 days in 6-well trays (3×10⁶ cells/well) supplemented with 1000 U GM-CSF per ml (Immunex, Seattle, Wash.) and 200 U IL-4 per ml (R&D Systems; Minneapolis, Minn.) at days 0, 2, 4 and 6.

### Generation of Mouse DCs

Bone marrow-derived DCs (BM-DC) were generated using modifications of the original method described by Inaba et al (42). In brief, bone marrow suspensions were incubated with red cell lysis buffer (PUREGENE™ RBC Lysis Solution, Gentra Systems, Minneapolis, Minn.) to remove red blood cells. After washing in media, lymphocytes and Ia-positive cells were killed with a cocktail of mAbs and rabbit complement for 60 min at 37° C. The mAbs were GK1.5 anti-CD4, TIB211 anti-CD8, TIB 120 anti-1a, and TIB 146 anti B220 (The Abs were kindly provided by Dr. Ralph Steinman). The cells were subsequently cultured in media containing 5% FCS and 10 ng/ml recombinant mouse GM-CSF (R&D Systems; Minneapolis, Minn.) for 7 days. For some experiments the cells were further purified at day 7 using CD11c⁺-microbeads (Miltenyi Biotech., Auburn, Calif.) according to the manufacturer's instructions.

### Stimulation of DCs

At day 7 of culture, immature DCs were either left untreated, or were stimulated with indicated doses of HMGB1-Bx, HMGB1 peptides, or LPS (E. coli serotype 026:B6, Sigma). In all experiments, DCs were analyzed 48 h after stimulation.

### Analysis of DC Phenotype

1×10⁴ DCs were reacted for at least 20 min at 4° C. in 100 μl of PBS/5% FCS/0.1% sodium azide (staining buffer) with fluorescein isothiocyanate (FITC)-conjugated IgG mAb specific for CD86, CD40 and MHC-II (eBioscience). Cells were then washed 4 times with staining buffer, fixed in 10% formaldehyde in PBS (pH 7.2-7.4) and examined by flow cytometry using a FACScan (BD). In all experiments, isotype controls were included using FITC-conjugated irrelevant mAb of the same Ig class.

### Measurement of Cytokines and Chemokines

48 h post activation, the production of cytokines and chemokines in cell culture supernatants was measured by ELISA (Pierce Boston Technology Center, SearchLight™ Proteome Arrays Multiplex Sample Testing Services, Woburn, Mass.).

### Mixed Leukocyte Reaction

To assess levels of T cell activation and proliferation, cells were plated at 10⁵ cells per well in a round-bottomed 96-well tray at DC:T cell ratios of 1:120 for 5 days in medium described above. The microcultures were pulsed with (³H)-thymidine (1 μCi/well) for the final 8 h of culture. Cell cultures were harvested onto glass fiber filters with an automated multiple sample harvester and the amount of isotope incorporation was determined by liquid scintillation β-emission. Responses are reported as mean cpm of thymidine incorporation by triplicate cultures (+/−SEM).

### Results

HMGB1 Peptides Induce Cytokine Secretion in Human DCs.

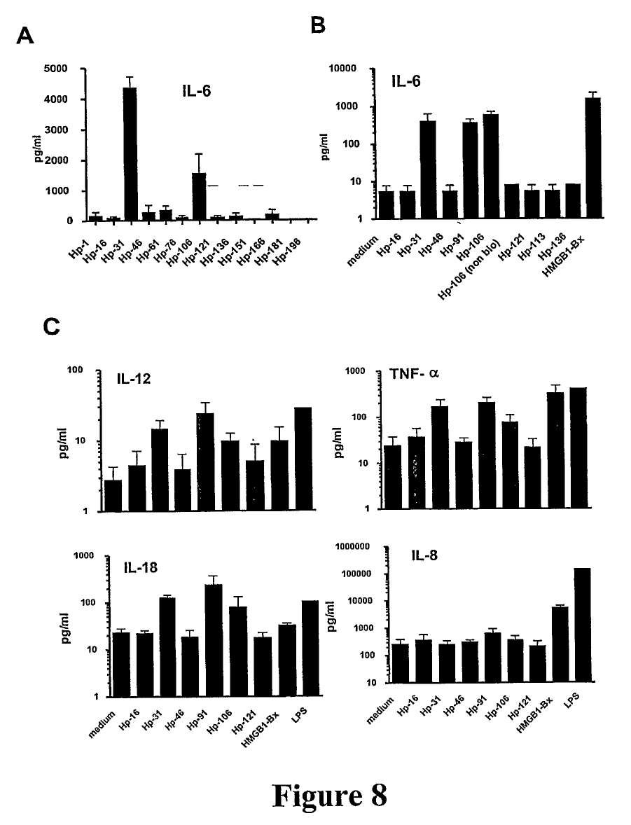

We have previously shown that a 18 amino acid long peptide whose sequence correspond to a part of the B box domain of HMGB1 induced IL-6 secretion in human monocyte-derived DCs (41). The search for DC activating peptides was extended by testing 18 amino acid long peptides that span the whole HMGB1 molecule (see Table 1). When human immature monocyte-derived DCs were exposed to these different peptides for 48 h, peptide Hp-31 in addition to the previously described peptide Hp-106 induced secretion of IL-6 by DCs (FIG. 8A). Subsequently peptides that overlap by three amino acids on either N- or C-terminus of these two peptides were tested (FIG. 8B). We found that the C-terminal flanking peptide Hp-91 also enhanced the IL-6 secretion and it shares only three amino acids (CSE) with Hp-106. The two peptides flanking Hp-31 had no activity. Furthermore, N-terminal biotinylation was required for the DC-stimulatory effect of the active peptides. The same sequence Hp-106 without N-terminal biotinylation (FIG. 8B, Hp-106-non bio) had no activity. However, the activity was not caused by biotin, since peptides with different sequences that were also N-terminally biotinylated, did not activate DCs (FIGS. 8A, 8B).

Next, the cytokine profile induced by the active peptide Hp-31 and Hp-106 and their flanking peptides was investigated. The three active peptides, Hp-31, Hp-91, and Hp-106, that induced IL-6 secretion also increased secretion of IL-12 (p 70), TNF-α, and IL-18, but not IL-8, whereas whole HMGB1-Bx enhanced production of IL-8 but not IL-18 (FIG. 8C and Table 2). Neither HMGB1-Bx nor the peptide-treated DCs showed enhanced secretion of IL-10, and TGF-β (Table 2 and data not shown).

TABLE 2
Cytokine profile in human and murine DCs stimulated
with HMGB1-Bx or HMGB1 peptides.

| HMG- | Hp- | Hp- | Hp- | Hp- |
|---|---|---|---|---|
| Bx | 16 | 31 | 91 | 106 |

HUMAN DC

| | | | | | |
|---|---|---|---|---|---|
| IL-6 | + | − | + | + | + |
| IL-12 | + | − | + | + | + |
| TNFα | + | − | + | + | + |
| IL-18 | − | − | + | + | + |
| IL-8 | + | − | − | − | − |
| IL-10 | − | − | − | − | − |
| IL-2 | − | n.a. | n.a. | n.a. | n.a. |
| IL-1β | − | n.a. | n.a. | n.a. | n.a. |
| IL-5 | n.a. | n.a. | n.a. | n.a. | n.a. |

Murine DC

| | | | | | |
|---|---|---|---|---|---|
| IL-6 | − | − | − | − | − |
| IL-12 | + | + | + | + | + |
| TNFα | + | − | n.a. | − | + |
| IL-18 | − | + | n.a. | − | + |
| IL-8 | + | − | n.a. | − | + |
| IL-10 | − | − | − | − | − |
| IL-2 | + | + | + | + | + |
| IL-1β | + | + | + | + | + |
| IL-5 | + | + | + | + | + |

n.a. = not analyzed, + indicates increase and − indicates no change compared to medium
The cytokine levels (pg/ml) were measure by ELISA 48 h after exposure to HMGB1-Bx or the peptides.

### HMGB1-Bx and HMGB1 Peptides Cause Secretion of Pro-Inflammatory Cytokines and Chemokines in Murine BM-DCs.

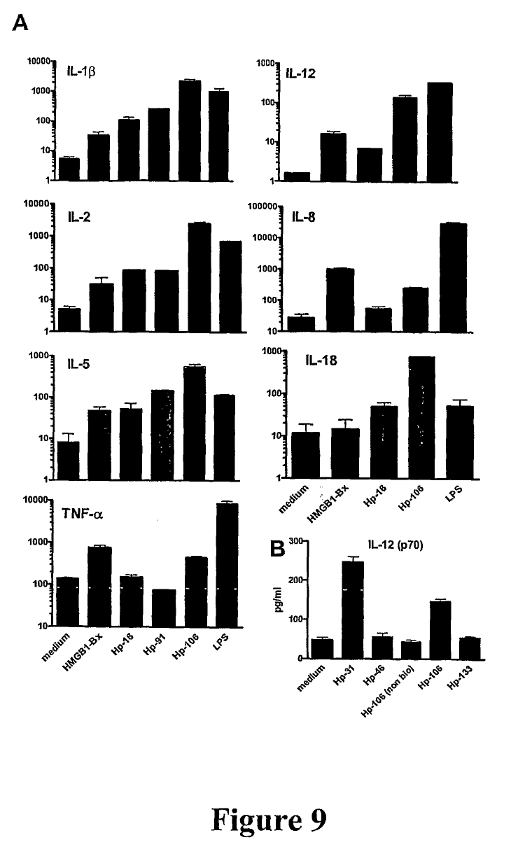

To determine whether HMGB1-Bx and HMGB1 peptides also enhance cytokine and chemokines secretion in murine DCs, immature bone marrow-derived DCs (BM-DCs) were exposed to HMGB1-Bx, HMGB1 peptides, or LPS for 48 h. HMGB1-Bx stimulated DCs had enhanced secretion of IL-1β, IL-2, IL-5, TNF-α, IL-12 (p70), and IL-8 but not IL-18 (FIG. 9A). In contrast to human DCs, HMGB1-Bx stimulated murine BM-DCs did not show enhanced secretion of IL-6 (Table 2). Murine BM-DCs were activated by the 3 peptides (Hp-106, Hp-91 and Hp31) that induced activation of human DCs, but also by peptide Hp-16 which had no effect on human DCs. Hp-16 and Hp-106 both enhanced secretion of IL-1β, IL-2, IL-5, IL-12, and IL-18, but only Hp-106 enhanced secretion of TNF-α and IL-8 (FIG. 9A). Interestingly, IL-18 production was enhanced by exposure of BM-DCs to the either of the two peptides but not to HMGB1-Bx. Hp-91, which enhanced cytokine secretion in human DCs also increased production of IL-1β, IL-2, and IL-5, but not of TNF-α (FIG. 9A), IL-18, and IL-8 in BM-DCs (Table 2 and data not shown).

Hp-31 enhanced the production of IL-12 (p70) (FIG. 9B), IL-2, IL-5, and IL-1β, but not IL-6 and IL-10 (Table 1) in murine BM-DCs. Furthermore, as observed in the human system N-terminal biotinylation was required. The non-biotinylated peptide (Hp-106 non-bio) that has the same sequence as Hp-106 did not enhance IL-12 secretion. Again, the DC stimulatory capacity of the peptides was sequence dependent, since biotinylated peptides with different sequences did not enhance IL-12 secretion (FIG. 9B).

### HMGB1 Peptides Induce Phenotypic Maturation of Murine BM-DCs.

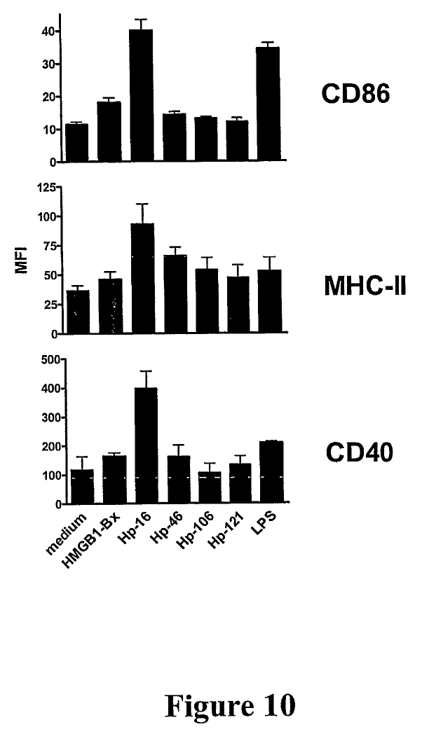

Previous work has linked the proinflammatory activity of HMGB1 to its B box domain (21). To determine whether the HMGB1-Bx and HMGB1 peptides in addition can induce phenotypic maturation of murine DCs, immature BM-DCs were exposed to HMGB1-Bx, HMGB1 peptides, or LPS for 48 h (FIG. 10). BM-DCs exposed to HMGB1-Bx showed only a small increase in CD86 expression and no changes in CD40 and MHC-II expression were observed. The Hp-16 peptide induced a strong upregulation of CD86, MHC-II, and CD40 to levels comparable to or higher than generated by LPS. Interestingly, although Hp-106 induced high levels of cytokine secretion in BM-DCs it did not significantly enhance the surface expression of maturation markers. No altered expression in MHC-11, CD86, or CD40 was detected using control peptide Hp-121.

### HMGB1-Bx and HMGB1 Peptides Induce Functional Maturation of BM-DCs.

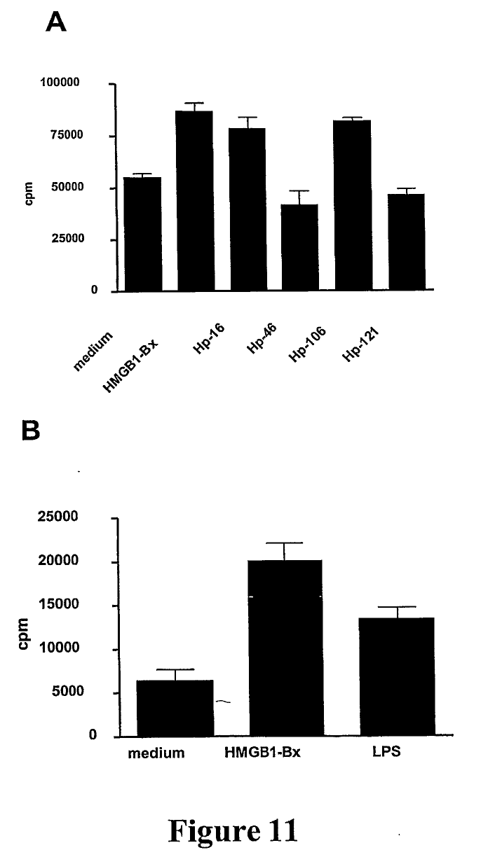

Mature, cytokine-producing DCs induce T cell activation and proliferation, leading to the development of adaptive immunity (1, 2, 22). To assess whether HMGB1-Bx and HMGB1 peptides induce functional maturation of BM-DCs, immature BM-DCs generated from C57/BL6 mice were exposed to HMGB1-Bx or HMGB1 peptides for 48 h and subsequently co-cultured with allogeneic T cells for 5 days. BM-DCs that were exposed to HMGB1-Bx, Hp-16 or Hp-106 activated resting allogeneic T cells in a mixed lymphocyte reaction (FIG. 11A), whereas DCs exposed to Hp-46 or Hp-121 did not show enhanced T cell stimulatory activity. To investigate whether the functional maturation of DCs caused by exposure to HMGB1-Bx was strain specific, BM-DCs were generated from Balb/c mice. HMGB1-Bx treated BM-DCs showed a strong capacity to induce T cell proliferation (FIG. 11B) as observed with BM-DCs generated from C57/BL6 mice.

### Summary

Collectively the data presented in the above examples demonstrate that HMGB1, its B box, and a number of distinct smaller peptides can function as maturation stimuli for human monocyte-derived immature DCs, and as such represent endogenous immunostimulatory molecules. Endogenous DC-stimulating factors are intriguing because they may represent a class of well-tolerated natural adjuvants (5). Hp-106 and to a lesser extent the Hp-16 act as Th1 stimuli by enhancing the production of IL-12 (p70), IL-2, and IL-18 in BM-DCs. The different spectra of cytokines and phenotypes that these peptides create in DCs might allow us to use peptides to custom tailor DCs with special features for therapeutic use. For example the IL-18 inducing capacity of the peptides is very attractive for cancer vaccine design as IL-18 together with IL-12 promotes anti tumor immune responses (43,44).

Biotinylation of the active peptides was necessary for the observed effects in both human and murine DCs. It is possible that biotinylation stabilizes the peptides. In fact is has been reported that introduction of biotin to the N-terminus of the insulin-like peptide promoted conformational stability which, in turn, allowed better receptor activation (45). Biotin-binding IgM has been detected in healthy subjects (46) and a biotin-binding protein has been detected in sera of female rats (47). It is conceivable that biotin binding proteins are present in the serum containing culture medium. Binding to these proteins could promote multimerization of the peptides and lead to receptor cross-linking, whereas non-biotinylated peptides might not be able to bind to the receptor due to their monomeric nature.

It has been shown that the A box domain of HMGB1 can inhibit HMGB1 activity and reverse established sepsis (49). Interestingly, two peptides whose sequence maps to the A box domain of HMGB1 (Hp-16 and Hp-31) have a stimulatory effect on both murine and human DCs. The Hp-16 peptide from A box induced lower levels of the cytokines but was the only one capable of inducing maturation. This could be due to different receptor usage and signaling pathways. It was tested whether peptides whose sequence maps to other regions within the A box domain could inhibit the cytokine-inducing capacity of Hp-31 by mixing the flanking peptides with Hp-31. None of the peptides tested affected the stimulatory capacity of Hp-31 (data not shown).

In summary, the selective activity of the HMGB1 peptides represents an attractive means to customize the functional properties of DCs in immunotherapeutic or vaccine context.

### REFERENCES

1. Banchereau, J., and R. M. Steinman. 1998. Dendritic cells and the control of immunity. Nature 392:245.
2. Rescigno, M., C. Winzler, D. Delia, C. Mutini, M. Lutz, and P. Ricciardi-Castagnoli. 1997. Dendritic cell maturation is required for initiation of the immune response. J Leukoc Biol 61:415.
3. De Smedt, T., B. Pajak, E. Muraille, L. Lespagnard, E. Heinen, P. De Baetselier, J. Urbain, O. Leo, and M. Moser. 1996. Regulation of dendritic cell numbers and maturation by lipopolysaccharide in vivo. J Exp Med 184:1413.
4. Hartmann, G., G. J. Weiner, and A. M. Krieg. 1999. CpG DNA: a potent signal for growth, activation, and maturation of human dendritic cells. Proceedings of the National Academy of Sciences of the United States of America 96:9305.
5. Gallucci, S., and P. Matzinger. 2001. Danger signals: SOS to the immune system. Current Opinion in Immunology 13:114.
6. Gallucci, S., M. Lolkema, and P. Matzinger. 1999. Natural adjuvants: endogenous activators of dendritic cells. Nature Medicine 5:1249.
7. Sauter, B., M. L. Albert, L. Francisco, M. Larsson, S. Somersan, and N. Bhardwaj.
2000. Consequences of cell death: exposure to necrotic tumor cells, but not primary tissue cells or apoptotic cells, induces the maturation of immunostimulatory dendritic cells. The Journal of Experimental Medicine 191:423.
8. Basu, S., R. J. Binder, R. Suto, K. M. Anderson, and P. K. Srivastava. 2000. Necrotic but not apoptotic cell death releases heat shock proteins, which deliver a partial maturation signal to dendritic cells and activate the NF-kappa B pathway. International Immunology 12:1539.
9. Goodwin, G. H., C. Sanders, and E. W. Johns. 1973. A new group of chromatin-associated proteins with a high content of acidic and basic amino acids. European Journal of Biochemistry 38:14.
10. Wang, H., O. Bloom, M. Zhang, J. M. Vishnubhakat, M. Ombrellino, J. Che, A. Frazier, H. Yang, S. Ivanova, L. Borovikova, K. R. Manogue, E. Faist, E. Abraham, J. Andersson, U. Andersson, P. E. Molina, N. N. Abumrad, A. Sama, and K. J. Tracey. 1999. HMG-1 as a late mediator of endotoxin lethality in mice. Science 285:248.
11. Andersson, U., H. Wang, K. Palmblad, A. C. Aveberger, O. Bloom, H. Erlandsson_Harris, A. Janson, R. Kokkola, M. Zhang, H. Yang, and K. J. Tracey. 2000. High mobility group 1 protein (HMG-1) stimulates proinflammatory cytokine synthesis in human monocytes. The Journal of Experimental Medicine 192:565.
12. Wang, H., H. Yang, C. J. Czura, A. E. Sama, and K. J. Tracey. 2001. HMGB1 as a late mediator of lethal systemic inflammation. American Journal of Respiratory and Critical Care Medicine: An Official Journal of the American Thoracic Society, Medical Section of the American Lung Association 164:1768.
13. Pullerits, R., I. M. Jonsson, M. Verdrengh, M. Bokarewa, U. Andersson, H. Erlandsson_Harris, and A. Tarkowski. 2003. High mobility group box chromosomal protein 1, a DNA binding cytokine, induces arthritis. Arthritis and Rheumatism 48:1693.
14. Abraham, E., J. Arcaroli, A. Carmody, H. Wang, and K. J. Tracey. 2000. HMG-1 as a mediator of acute lung inflammation. Journal of Immunology 165:2950.
15. Scaffidi, P., T. Misteli, and M. E. Bianchi. 2002. Release of chromatin protein HMGB1 by necrotic cells triggers inflammation. Nature 418:191.
16. Wang, H., J. M. Vishnubhakat, O. Bloom, M. Zhang, M. Ombrellino, A. Sama, and K. J. Tracey. 1999. Proinflammatory cytokines (tumor necrosis factor and interleukin 1) stimulate release of high mobility group protein-1 by pituicytes. Surgery 126:389.
17. Bustin, M., D. A. Lehn, and D. Landsman. 1990. Structural features of the HMG chromosomal proteins and their genes. Biochimica Et Biophysica Acta 1049:231.
18. Bustin, M., and R. Reeves. 1996. High-mobility-group chromosomal proteins: architectural components that facilitate chromatin function. Progress in Nucleic Acid Research and Molecular Biology 54:35.
19. Yang, H., H. Wang, C. J. Czura, and K. J. Tracey. 2002. HMGB1 as a cytokine and therapeutic target. J. Endotoxin Res. 8:469.
20. Kokkola, R., E. Sundberg, A. K. Ulfgren, K. Palmblad, J. Li, H. Wang, L. Ulloa, H.
Yang, X. J. Yan, R. Furie, N. Chiorazzi, K. J. Tracey, U. Andersson, and H. E. Harris.
2002. High mobility group box chromosomal protein 1: a novel proinflammatory mediator in synovitis. Arthritis and Rheumatism 46:2598.
21. Li, J., R. Kokkola, S. Tabibzadeh, R. Yang, M. Ochani, X. Qiang, H. E. Harris, C. J. Czura, H. Wang, L. Ulloa, H. S. Warren, L. L. Moldawer, M. P. Fink, U. Andersson, K. J. Tracey, and H. Yang. Structural basis for the proinflammatory cytokine activity of high mobility group box 1. Molecular Medicine 9:37.
22. Rescigno, M., C. Winzler, D. Delia, C. Mutini, M. Lutz, and P. Ricciardi_Castagnoli.
1997. Dendritic cell maturation is required for initiation of the immune response. Journal of Leukocyte Biology 61:415.
23. Hori, O., J. Brett, T. Slattery, R. Cao, J. Zhang, J. X. Chen, M. Nagashima, E. R. Lundh, S. Vijay, and D. Nitecki. 1995. The receptor for advanced glycation end products (RAGE) is a cellular binding site for amphoterin. Mediation of neurite outgrowth and co-expression of rage and amphoterin in the developing nervous system. The Journal of Biological Chemistry 270:25752.
24. Huttunen, H. J., C. Fages, and H. Rauvala. 1999. Receptor for advanced glycation end products (RAGE)-mediated neurite outgrowth and activation of NF-kappaB require the cytoplasmic domain of the receptor but different downstream signaling pathways. The Journal of Biological Chemistry 274:19919.
25. Clark, G. J., S. Gunningham, A. Troy, S. Vuckovic, and D. N. Hart. 1999. Expression of the RelB transcription factor correlates with the activation of human dendritic cells. Immunology 98:189.
26. Neumann, M., H. Fries, C. Scheicher, P. Keikavoussi, A. Kolb-Maurer, E. Brocker, E. Serfling, and E. Kampgen. 2000. Differential expression of Rel/NF-kappaB and octamer factors is a hallmark of the generation and maturation of dendritic cells. Blood 95:277.
27. Rescigno, M., M. Martino, C. L. Sutherland, M. R. Gold, and P. Ricciardi-Castagnoli. 1998. Dendritic cell survival and maturation are regulated by different signaling pathways. J Exp Med 188:2175.
28. Messmer, D., J. Bromberg, G. Devgan, J. M. Jacque, A. Granelli-Pipemo, and M. Pope. 2002. Human immunodeficiency virus type 1 Nef mediates activation of STAT3 in immature dendritic cells. AIDS Res Hum Retroviruses 18:1043.
29. Park, J. S., D. Svetkauskaite, Q. He, J. Y. Kim, D. Strassheim, A. Ishizaka, and E. Abraham. 2004. Involvement of toll-like receptors 2 and 4 in cellular activation by high mobility group box 1 protein. The Journal of Biological Chemistry 279:7370.
30. Ouaaz, F., M. Li, and A. A. Beg. 1999. A critical role for the RelA subunit of nuclear factor kappaB in regulation of multiple immune-response genes and in Fas-induced cell death. The Journal of Experimental Medicine 189:999.
31. Li, M., D. F. Carpio, Y. Zheng, P. Bruzzo, V. Singh, F. Ouaaz, R. M. Medzhitov, and A. A. Beg. 2001. An essential role of the NF-kappa B/Toll-like receptor pathway in induction of inflammatory and tissue-repair gene expression by necrotic cells. Journal of Immunology 166:7128.
32. Aicher, A., G. L. Shu, D. Magaletti, T. Mulvania, A. Pezzutto, A. Craxton, and E. A. Clark. 1999. Differential role for p38 mitogen-activated protein kinase in regulating CD40-induced gene expression in dendritic cells and B cells. J Immunol 163:5786.
33. Sato, K., H. Nagayama, K. Tadokoro, T. Juji, and T. A. Takahashi. 1999. Extracellular signal-regulated kinase, stress-activated protein kinase/c-Jun N-terminal kinase, and p38mapk are involved in IL-10-mediated selective repression of TNF-alpha-induced activation and maturation of human peripheral blood monocyte-derived dendritic cells. J Immunol 162:3865.
34. Arrighi, J. F., M. Rebsamen, F. Rousset, V. Kindler, and C. Hauser. 2001. A critical role for p38 mitogen-activated protein kinase in the maturation of human blood-derived dendritic cells induced by lipopolysaccharide, TNF-alpha, and contact sensitizers. J Immunol 166:3837.
35. Erlandsson Harris, H. and Andersson, U., Mini-review: The nuclear protein HMGB1 as a proinflammatory mediator. Eur J Immunol 2004. 34: 1503-1512.
36. Demarco, R. A., Fink, M. P. and Lotze, M. T., Monocytes promote natural killer cell interferon gamma production in response to the endogenous danger signal HMGB1. Mol Imnunol 2005.42: 433-444.
37. Kokkola, R., Andersson, A., Mullins, G., Ostberg, T., Treutiger, C. J., Arnold, B., Nawroth, P., Andersson, U., Harris, R. A. and Harris, H. E., RAGE is the major receptor for the proinflammatory activity of HMGB1 in rodent macrophages. Scand J Immunol 2005. 61: 1-9.
39. Andersson, U. and Erlandsson-Harris, H., HMGB1 is a potent trigger of arthritis. J Intern Med 2004. 255: 344-350.
40. Li, J., Kokkola, R., Tabibzadeh, S., Yang, R., Ochani, M., Qiang, X., Harris, H. E., Czura, C. J., Wang, H., Ulloa, L., Warren, H. S., Moldawer, L. L., Fink, M. P., Andersson, U., Tracey, K. J. and Yang, H., Structural basis for the proinflammatory cytokine activity of high mobility group box 1. Molecular Medicine 9: 37-45.
41. Messmer, D., Yang, H., Telusma, G., Knoll, F., Li, J., Messmer, B., Tracey, K. J. and Chiorazzi, N., High mobility group box protein 1: an endogenous signal for dendritic cell maturation and Th1 polarization. J Immunol 2004. 173: 307-313.
42. Inaba, K., Inaba, M., Romani, N., Aya, H., Deguchi, M., Ikehara, S., Muramatsu, S, and Steinman, R. M., Generation of large numbers of dendritic cells from mouse bone marrow cultures supplemented with granulocyte/macrophage colony-stimulating factor. The Journal of Experimental Medicine 1992. 176: 1693-1702.
43. Osaki, T., Hashimoto, W., Gambotto, A., Okamura, H., Robbins, P. D., Kurimoto, M., Lotze, M. T. and Tahara, H., Potent antitumor effects mediated by local expression of the mature form of the interferon-gamma inducing factor, interleukin-18 (IL-18). Gene Ther 1999. 6: 808-815.
44. Tahara, H. and Lotze, M. T., Antitumor effects of interleukin-12 (IL-12): applications for the immunotherapy and gene therapy of cancer. Gene Ther 1995. 2: 96-106.
45. Fu, P., Layfield, S., Ferraro, T., Tomiyama, H., Hutson, J., Otvos, L., Jr., Tregear, G. W., Bathgate, R. A. and Wade, J. D., Synthesis, conformation, receptor binding and biological activities of monobiotinylated human insulin-like peptide 3. J Pept Res 2004. 63: 91-98.
46. Nagamine, T., Takehara, K., Fukui, T. and Mori, M., Clinical evaluation of biotin-binding immunoglobulin in patients with Graves' disease. Clin Chim Acta 1994. 226: 47-54.
47. Seshagiri, P. B. and Adiga, P. R., Isolation and characterisation of a biotin-binding protein from the pregnant-rat serum and comparison with that from the chicken egg-yolk. Biochim Biophys Acta 1987. 916: 474-481.
48. Messmer, D., Jacque, J. M., Santisteban, C., Bristow, C., Han, S. Y., Villamide-Herrera, L., Mehlhop, E., Marx, P. A., Steinman, R. M., Gettie, A. and Pope, M., Endogenously expressed nef uncouples cytokine and chemokine production from membrane phenotypic maturation in dendritic cells. J Immunol 2002.169: 4172-4182.
49. Yang, H., Ochani, M., Li, J., Qiang, X., Tanovic, M., Harris, H. E., Susarla, S. M., Ulloa, L., Wang, H., DiRaimo, R., Czura, C. J., Roth, J., Warren, H. S., Fink, M. P., Fenton, M. J., Andersson, U. and Tracey, K. J., Reversing established sepsis with antagonists of endogenous high-mobility group box 1. Proc Natl Acad Sci USA 2004. 101: 296-301.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes. In addition, U.S. Provisional Application No. 60/580,549, filed Jun. 17, 2004, is incorporated by reference in its entirety for all purposes.

1. An immunogenic composition comprising a fusion polypeptide, wherein said fusion polypeptide comprises an HMGB1 polypeptide or a functional variant thereof fused to a heterologous antigen.

2. The immunogenic composition of claim 1, wherein said HMGB1 polypeptide is selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20 and SEQ ID NO:21.

3. The immunogenic composition of claim 1, wherein said HMGB1 polypeptide is a B box polypeptide or a biologically active fragment thereof.

4. (canceled)

5. (canceled)

6. The immunogenic composition of claim 3, wherein the HMGB1 B box biologically active fragment comprises the amino acid sequence of SEQ ID NO:12 or SEQ ID NO: 13.

7. (canceled)

8. (canceled)

9. The immunogenic composition of claim 1, further comprising a carrier, or an adjuvant, or a carrier and an adjuvant.

10. A method of vaccinating an animal comprising administering the immunogenic composition of claim 1.

11. A method of stimulating or increasing an immune response in an individual in need of immunostimulation, said method comprising administering the immunogenic composition of claim 1 in an amount sufficient to stimulate or increase said immune response.

12. The method of claim 11, wherein inflammation is reduced as compared to administering said heterologous antigen and said HMGB1 B polypeptide separately (i.e., unfused).

13. A method of activating APCs comprising administering the immunogenic composition of claim 1.

14. (canceled)

15. An isolated polynucleotide encoding the fusion polypeptide of claim 1.

16. The immunogenic composition of claim 1, wherein the antigen is a tumor, bacterial or viral antigen.

17. A method of treating or preventing cancer comprising administering the immunogenic composition of claim 1.

18. The immunogenic composition of claim 1, wherein said immunogenic composition activates APCs.

19. A method of stimulating or increasing an immune response in an individual in need of immunostimulation, said method comprising administering the immunogenic composition of claim 18 in an amount sufficient to stimulate or increase said immune response.

20. The immunogenic composition of claim 18, wherein the HMGB1 polypeptide is a HMGB1 B box or a biologically active fragment thereof.

21. The immunogenic composition of claim 20, wherein the HMGB1 B box biologically active fragment comprises the amino acid sequence of SEQ ID NO:12 or SEQ ID NO: 13.

22. (canceled)

23. An isolated cell that produces a fusion polypeptide, wherein said fusion polypeptide comprises an HMGB1 polypeptide or a functional variant thereof fused to a heterologous antigen.

24. The isolated cell of claim 23, wherein said HMGB1 polypeptide is selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO: 16, SEQ ID NO: 17, SEQ ID NO: 18, SEQ ID NO: 19, SEQ ID NO:20 and SEQ ID NO:21.

25. The isolated cell of claim 23, wherein said HMGB1 polypeptide is a B box polypeptide or a biologically active fragment thereof.

26. The isolated cell of claim 25, wherein the HMGB1 B box biologically active fragment comprises the amino acid sequence of SEQ ID NO:12 or SEQ ID NO: 13.

27. (canceled)
